(19) United States
(12) Patent Application Publication
(10) Pub. No.: US 2020/0303534 A1
(43) Pub. Date: Sep. 24, 2020

(54) MICROELECTRONIC SENSOR WITH AN AHARONOV-BOHM ANTENNA

(71) Applicant: EPITRONIC HOLDINGS PTE. LTD., Singapore (SG)

(72) Inventors: Ayal RAM, Singapore (SG); Walid-Madhat MUNIEF, Blieskastel (DE)

(21) Appl. No.: 16/808,898

(22) Filed: Mar. 4, 2020

Related U.S. Application Data
(60) Provisional application No. 62/821,896, filed on Mar. 21, 2019.

(51) Int. Cl. H01L 29/778; A61B 5/05; H01L 29/417; H01L 29/45; A61B 5/145; H01L 29/20

(52) U.S. Cl. CPC H01L 29/7786; A61B 5/0507; H01L 29/4175; A61B 2562/028; A61B 5/14542; H01L 29/2003; H01L 29/454

(57) ABSTRACT

The present invention describes a microelectronic sensor based on the combination of an open-gate pseudo-conductive high-electron mobility transistor and a metamaterial electrode installed in the open gate area of the transistor and operated in the sub-THz and THz frequency range. The sensor can be used in different applications, for example, chemical sensing and biomolecular diagnostics, monitoring glucose levels in blood and biometric authentication of a user.

### CROSS REFERENCE TO RELATED APPLICATIONS

This application claims the benefit of and priority to U.S. Provisional Application No. 62/821,896 filed Mar. 21, 2019 entitled MICROELECTRONIC SENSOR WITH AN AHARONOV-BOHM ANTENNA. The contents of the above application is incorporated by reference as if fully set forth herein in its entirety.

### TECHNICAL FIELD

The present application relates to the field of microelectronic sensors based on pseudo-conducting high-electron-mobility transistors incorporating a metamaterial electrode and their use in detection and continuous monitoring of electrical signals in a sub-THz and THz range.

### BACKGROUND

### High-Electron Mobility Transistors

The polarization doped high-electron-mobility transistor (HEMT) is a field effect transistor (FET) in which two layers of different bandgap and polarisation field are grown upon each other forming a hetero junction structure. As a consequence of the discontinuity in the polarisation field, surface charges are created at the interface between the layers of the hetero junction structure. If the induced surface charge is positive, electrons will tend to compensate the induced charge resulting in the formation of the channel Since in the HEMT, the channel electrons are confined in a quantum well in an infinitely narrow spatial region at the interface between the layers, these electrons are referred to as a two-dimensional electron gas (2DEG). This special confinement of the channel electrons in the quantum well actually grants them two-dimensional features, which strongly enhance their mobility surpassing the bulk mobility of the material in which the electrons are flowing.

The HEMTs based on the layers of III-V semiconductor materials, such as gallium nitride (GaN) and aluminium gallium nitride (AlGaN), have recently been developed with a view to high-voltage and high-power switching applications. The high voltages and high switching speeds allow smaller, more efficient devices, such as home appliances, communications and automobiles to be manufactured. To control the density of electrons in the 2DEG channel and to switch the HEMT on and off, the voltage at the gate of the transistor should be regulated.

FIGS. 1a-1c schematically shows the quantum well at three different biasing conditions starting from the positive gate potential (V_{G}), much higher than the threshold voltage (VT), and going down to the 0V gate potential and further to the negative values below the threshold voltage. The V_{T} is defined as a voltage required to populate electrons at the interface between the GaN and AlGaN layers, thereby creating conductivity of the 2DEG channel Since the 2DEG channel electrons occupy energy levels below the Fermi level, the Fermi level in a quantum well is located above several energy levels when V_{G}>>V_{T} (FIG. 1a). This enables high population of the 2DEG channel electrons and hence, high conductivity. The HEMT is turned on in this case. However, when V_{G} decreases to 0V (FIG. 1b), the Fermi level also drops with respect to the quantum well. As a result, much fewer electron energy levels are populated and the amount of the 2DEG channel electrons significantly decreases. When V_{G}<<V_{T} (FIG. 1c), all electron energy levels are above the Fermi level, and there is no 2DEG electrons below the gate. This situation is called “channel depletion”, and the HEMT is turned off.

Many commercially available AlGaN/GaN-based HEMT structures have a negative V_{T}, resulting in a “normally-on” operation mode at 0V gate potential. They are called “depletion-mode transistors” and used in various power switching applications when the negative voltage must be applied on the gate in order to block the current. However, for safe operation at high voltage or high power density, in order to reduce the circuit complexity and eliminate standby power consumption, HEMTs with “normally-off” characteristics are preferred.

Several techniques to manufacture the normally-off HEMTs have been reported. Burnham et al in “Gate-recessed normally-off GaN-on-Si HEMT using a new O₂—BCl₃ digital etching technique”, Phys. Status Solidi C, Vol. 7, 2010, No. 7-8, pp. 2010-2012, proposed normally-off structures of the recessed gate type. In this structure, the AlGaN barrier layer is etched and the gate is brought closer to the interface between the AlGaN barrier layer and the GaN buffer layer. As the gate approaches the interface between the layers, the V_{T} increases. The normally-off operation of the transistor is achieved once the depletion region reaches the interface and depletes the 2DEG channel at zero gate voltage. The major advantages of these HEMTs are lower power consumption, lower noise and simpler drive circuits. These HEMTs are currently used, for example, in microwave and millimetre wave communications, imaging and radars.

Chang et al in “Development of enhancement mode AlN/GaN high electron mobility transistors”, Appl. Phys. Lett., Vol. 94, 2009, No. 26, p. 263505, proposed using a very thin AlGaN barrier instead of etching the relatively thick barrier layer to approach the AlGaN/GaN interface. This structure also achieves the normally-off operation by approaching the transistor gate towards the AlGaN/GaN interface. Chen et al (2010) in “Self-aligned enhancement-mode AlGaN/GaN HEMTs using 25 keV fluorine ion implantation”, in Device Research Conference (DRC), 2010, pp. 137-138, proposed to use the fluorine-based plasma treatment method. Although many publications have adopted various methods to achieve normally-off devices with minimum impact on the drain current, they unfortunately sacrificed device turn-on performance.

### Aharonov-Bohm Effect

Iconic introductions to quantum mechanics by Richard Feynman emphasise interference as “mysterious behaviour at the heart of quantum mechanics” and mention that “any other situation in quantum mechanics, it turns out, can always be explained by saying ‘You remember the case of the experiment with the two holes? It's the same thing.’ This central mystery has been the subject of numerous direct experimental tests for the past several decades. This is also the case with the Aharonov-Bohm effect, wherein the presence of a magnetic field within the barrier between the two slits affects the interference pattern, despite the fact that the particle is rigorously excluded from that barrier.

In general, the quantum phenomenon, which was once considered to limit the possibilities of measurements, is now extensively used to improve them and increase sensitivity of nanometre-scale sensor devices. Unfortunately, the Aharonov-Bohm effect discovered by Yakir Aharonov and David Bohm in 1959 is not a good example of that progress. Although this effect possibly constitutes a modern cornerstone of quantum mechanics, until now its origin is still unclear and its understanding remain far from complete.

To understand the phenomenon discovered by Aharonov and Bohm, the following question should be posed: What happens to an electron as it passes nearby an infinitely long ideal solenoid (in a magnetic field that creates a solenoid and surrounded by a region completely free of electromagnetic field)? The obvious answer would be ‘nothing’, since outside the solenoid, the magnetic and electric fields (and consequently, the Lorentz forces) do not exist, and the electron is therefore not supposed to feel any disturbances. This is the classical physics point of view, where the electron moves in this free region and does not experience any type of magnetic force. But in quantum physics, when the electron orbits around the solenoid, its wave function experiences a phase shift proportional to the flux of the magnetic field even though the field is zero in the region where the particle passes through. This effect discovered by Aharonov and Bohm is a pure quantum-mechanical interaction, which is not originated from forces, but from potentials described by the Hamiltonian. It is a result of the fact that the wave function is a gauge dependent quantity, i.e. it depends on the electromagnetic vector potential, which is a non-unique, non-physical quantity.

Aharonov and Bohm then suggested that any electrically charged particle is influenced by the vector potential in regions in which the magnetic field B=∇×A is zero. A beam of electrons at the same energy level passes through a double slit on opposite sides of a solenoid. The expected interference pattern of the waves going through the two slits is shifted by an additional phase difference when the solenoid encloses a magnetic field, despite the magnetic field being zero in all the regions through which the electrons pass. This situation is illustrated in FIG. 2 showing a solenoid having an electric current inside and placed just behind the slits. Uniform magnetic field is present only inside the solenoid (the magnetic field outside the solenoid is zero), but the vector potential of the magnetic field is non-zero outside. This can be observed experimentally by the horizontal displacement of the interference fringes. This effect occurs in small disordered metallic conductors and causes conductance fluctuations in small (non-superconducting) rings and wires. It arises from the presence of a vector potential produced by an applied magnetic field. A potential is itself a fundamental physical entity, and would affect a charged particle even in a region in which there was no magnetic field, and therefore no force acting on the charged particle.

The experiment proposed by Aharonov and Bohn and shown in FIG. 2 clearly supports the existence of the effect. If a current is applied to a solenoid of infinite length, the magnetic field would not exist outside the solenoid, but there would be a vector potential. If one beam of electrons passes on one side of the solenoid and another beam of electrons passes on the other side of the solenoid, the phase difference between the two beams would be proportional to the magnetic flux inside the solenoid, even though neither electron beam would be subjected to a magnetic field. This phenomenon is referred to as the Aharonov-Bohm effect, and was the subject of many fierce debates since it related to a fundamental of physics. Those who accepted this interpretation of the observed phenomenon insisted that vector potential physically influences electrons, while those who argued against the existence of the Aharonov-Bohm effect claimed that vector potential was a mere mathematical entity.

Tonomura et al in “Observation of Aharonov-Bohm Effect by Electron Holography”, Phys. Rev. Lett. 48, 1982, p. 1443-1446, showed the measurements (using a holography electron microscope) of the phase difference in the form of interference fringes produced by two beams of electrons, one passing through the inside and the other passing through the outside of a doughnut-shaped ferromagnet. They clearly indicated the presence of a phase difference between the two electrons beams passing through a space where there is no magnetic field, and that the extent of the phase difference precisely matches the predicted value.

Later, to overcome the problem of leakage magnetic fields in space (from the solenoid to the electron beams contacting the solenoid) that might create the observed phase difference, Tonomura et al in “Evidence for Aharonov-Bohm effect with magnetic field completely shielded from electron wave”, Phys. Rev. Lett. 56, 1986, 792, resorted to a microfabrication technique developed to produce semiconductor devices. Osakabe et al in “Experimental confirmation of Aharonov-Bohm effect using a toroidal magnetic field confined by a superconductor”, Phys. Rev. A 34, 1986, 815, described fabrication of a doughnut-shaped (toroidal) ferromagnet, six microns in diameter, and covered it with a niobium superconductor to completely confine the magnetic field within the doughnut, which is in accordance with the Meissner effect. With the magnet maintained at 5 K, they measured the phase difference from the interference fringes between one electron beam passing through the hole in the doughnut and the other passing on the outside of the doughnut. The resulting interference fringes were successfully displaced with just half a fringe of spacing inside and outside of the doughnut, indicating the existence of the effect.

Thus, the Aharonov-Bohm effect has changed our notion of electromagnetic field and potential. It is known as a milestone in our understanding of electromagnetic interactions, which describes a quantum interference of a charged particle moving in a region free of electric and magnetic fields. The general consensus is that the Aharonov-Bohm effect demonstrates the invalidity of the classical picture of electromagnetism based on the local action of fields. Although electrons pass through regions free of any electromagnetic field, an observable effect was produced due to the existence of vector potentials. In this way, the long dispute on the Aharonov-Bohm effect was brought to a close by this one picture.

### SUMMARY

The present invention relates to a an open-gate pseudo-conductive high-electron mobility transistor comprising:
(i) a multilayer heterojunction structure being composed of III-V single-crystalline or polycrystalline semiconductor materials and deposited on a substrate layer (10) or placed on free-standing membranes (18), said structure comprising at least one buffer layer (11) and at least one barrier layer (12), said layers being stacked alternately;
(ii) a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current, respectively, in said transistor between source and drain contacts (15);
(iii) the source and drain contacts (15) connected to said 2DEG or 2DHG conducting channel (13) and to electrical metallisations (14) for connecting said transistor to an electric circuit; and
(iv) a metamaterial electrode (16) placed on a top layer of said heterojunction structure between said source and drain contacts (15) in the open gate area (17) of the transistor, said metamaterial electrode (16) is capable of detecting electrical signals in the sub-THz or THz frequency range between 30 GHz to 300 THz;
characterised in that the thickness (d) of the top layer of said heterojunction structure in the open gate area (17) is 5-9 nanometres (nm) which corresponds to the pseudo-conducting current range between normally-on and normally-off operation mode of the transistor, and the surface of said top layer has a roughness of about 0.2 nm or less, wherein the combination of said thickness and said roughness of the top layer allows to observe the pseudo-conducting current in said transistor.

In some embodiments, the source and drain contacts of are ohmic. In other embodiments, the electrical metallisations are capacitively-coupled to the 2DEG or 2DHG conducting channel for inducing displacement currents, thus resulting in said source and drain contacts being non-ohmic. In a particular embodiment, the transistor further comprises a dielectric layer deposited on top of the multilayer hetero junction structure.

In a specific embodiment, said III-V single-crystalline or polycrystalline semiconductor materials are GaN/AlGaN, and said heterojunction structure comprises either:
(a) (i) one top AlGaN layer recessed in an open gate area of the transistor to the thickness of 5-9 nm and having the surface roughness of 0.2 nm or less, and (ii) one bottom GaN buffer layer; said layers have Ga-face polarity, thus forming the two-dimensional electron gas (2DEG) conducting channel in said GaN layer, close to the interface with said AlGaN layer; or
(b) (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have Ga-face polarity, thus forming a two-dimensional hole gas (2DHG) conducting channel in the top GaN layer, close to the interface with said AlGaN barrier layer; or
(c) (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have N-face polarity, thus forming a two-dimensional electron gas (2DEG) conducting channel in the top GaN layer, close to the interface with said AlGaN barrier layer; or
(d) (i) one top AlGaN layer recessed in an open gate area of the transistor to the thickness of 5-9 nm and having the surface roughness of 0.2 nm or less, and (ii) one bottom GaN buffer layer; said layers have N-face polarity, thus forming a two-dimensional hole gas (2DHG) conducting channel in the GaN buffer layer, close to the interface with said AlGaN barrier layer.

In another specific embodiment, the thickness of the top layer (GaN or AlGaN) of the transistor in the open gate area is 6-7 nm, mores specifically 6.2 nm to 6.4 nm, and the surface of said top layer has a roughness of 0.2 nm or less, more specifically 0.1 nm or less, or 0.05 nm or less.

In still another embodiment, the transistor of the present invention further comprises at least one molecular or biomolecular layer immobilised within the open gate area of the transistor and capable of binding or adsorbing target (analyte) gases, chemical compounds or biomolecules from the environment. Said molecular or biomolecular layer is selected from a cyclodextrin, 2,2,3,3-tetrafluoropropyloxy-substituted phthalocyanine or their derivatives, or said molecular or biomolecular layer comprises capturing biological molecules, such as primary, secondary antibodies or fragments thereof against certain proteins to be detected, or their corresponding antigens, enzymes or their substrates, short peptides, specific DNA sequences, which are complimentary to the sequences of DNA to be detected, aptamers, receptor proteins or molecularly imprinted polymers.

Another aspect of the present invention is a microelectronic sensor for sensing electrical signals in the frequency range between 30 GHz to 300 THz, comprising either (i) at least one single transistor of the present invention, or at least one pair of the transistors of the present invention installed within said sensor with their open gates facing each other and thereby forming a resonant cavity enclosed by their open gates, said resonant cavity is capable of maintaining resonance in the frequency range between 30 GHz to 300 THz.

In still another aspect of the present invention, a microelectronic sensor for sensing electrical signals in the frequency range between 30 GHz to 300 THz comprises:
(a) a DC/RF-based antenna array of the transistors of any one of claims 1 to 10, wherein each transistor in said array is connected to its dedicated electrical contact line;
(b) a row multiplexer connected to the array for addressing a plurality of said transistors arranged in rows, selecting one of several analogue or digital input signals and forwarding the selected input into a single line; and
(c) a column multiplexer connected to said array for addressing a plurality of said transistors arranged in columns, selecting one of several analogue or digital input signals and forwarding the selected input into a single line.

In a further embodiment, the microelectronic sensors of the present invention further comprise:
(a) an integrated circuit for storing and processing signals in the frequency range between 30 GHz to 300 THz, and for modulating and demodulating radio-frequency (RF) signals;
(b) an μ-pulse generator for pulsed RF signal generation;
(c) an integrated DC-RF current amplifier or lock-in amplifier connected to said μ-pulse generator for amplification of the signal obtained from said μ-pulse generator;
(d) an analogue-to-digital converter (ADC) with in-built digital input/output card connected to the amplifier for converting the received analogue signal to a digital signal and outputting said digital signal to a microcontroller unit;
(e) the microcontroller unit (MCU) for processing and converting the received digital signal into data readable in a user interface or external memory; and
(f) a wireless connection module for wireless connection of said microelectronic sensor to said user interface or external memory.

In a certain aspect of the present invention, a method for chemical sensing and biomolecular diagnostics comprises:
A. Subjecting a sample to be tested to one of the microelectronic sensors of the present invention;
B. Recording electrical signals in the frequency range between 30 GHz to 300 THz received from the sample with said microelectronic sensor in a form of a source-drain electric potential of the microelectronic sensor over time (V_{DS} dynamics) or measuring S11-S12 parameters of the microelectronic sensor over time (S11-S12 dynamics);
C. Transmitting the recorded signals from said microelectronic sensor to an external memory for further processing; and
D. Converting the transmitted signals to digital signals and processing the digital signals in the external memory, comparing said V_{DS} dynamics or S11-S12 dynamics with negative control chemical or biomolecular V_{DS} or S11-S12-transfer waveforms stored in the external memory, and extracting chemical or biomolecular information from said waveforms in a form of readable data, thereby detecting and identifying a particular chemical or biological compound in the sample and measuring its concentration.

In a further aspect of the present invention, a method for non-invasive monitoring of glucose levels in blood comprises:
A. Contacting a single sensing point on the user's body with, or remotely positioning in a space against the user's body, one of the microelectronic sensors of the present invention;
B. Recording electrical signals received from the user's body with the microelectronic sensor in a form of a source-drain electric potential of the microelectronic sensor over time (V_{DS} dynamics) or measuring S11-S12 parameters of the microelectronic sensor over time (S11-S12 dynamics);
C. Transmitting the recorded signals from said microelectronic sensor to an external memory for further processing; and
D. Converting the transmitted signals to digital signals and processing the digital signals in the external memory, correlating said V_{DS} dynamics or S11-S12 dynamics with pre-calibrated waveforms for blood glucose levels stored in the external memory, and calculating the user's blood glucose levels from said signals in a form of a numerical or readable data, thereby monitoring the blood glucose levels of the user.

In yet further aspect of the present invention, a method for biometric authentication of a user comprises:
A. Contacting a single sensing point on the user's body with the microelectronic sensor of any one of claims 11 to 13, remotely positioning said sensor in a space against the user's body, or activating said sensor on calling or when the contact is established;
B. Recording electrical signals received from the user's body with the microelectronic sensor in a form of a source-drain electric potential of the microelectronic sensor over time (V_{DS} dynamics) or measuring S11-S12 parameters of the microelectronic sensor over time (S11-S12 dynamics);
C. Transmitting the recorded signals from said microelectronic sensor to an external memory for further processing; and
D. Converting the transmitted signals to digital signals and processing the digital signals in the external memory, and comparing said V_{DS} dynamics or S11-S12 dynamics with pre-calibrated biometric data of the user stored in the external memory, thereby biometrically authenticating the user.

Various embodiments may allow various benefits and may be used in conjunction with various applications. The details of one or more embodiments are set forth in the accompanying figures and the description below. Other features, objects and advantages of the described techniques will be apparent from the description and drawings and from the claims

### BRIEF DESCRIPTION OF THE DRAWINGS

The patent or application file contains at least one drawing executed in color. Copies of this patent or patent application publication with color drawings will be provided by the Office upon request and payment of the necessary fee. Disclosed embodiments will be understood and appreciated more fully from the following detailed description taken in conjunction with the appended figures. The drawings included and described herein are schematic and are not limiting the scope of the disclosure. It is also noted that in the drawings, the size of some elements may be exaggerated and, therefore, not drawn to scale for illustrative purposes. The dimensions and the relative dimensions do not necessarily correspond to actual reductions to practice of the disclosure.

FIGS. 1a-1c schematically shows the quantum well at three different biasing conditions:

FIG. 1a: positive gate potential (+VG) is much higher than threshold voltage (VT),

FIG. 1b: 0V gate potential, and

FIG. 1c: negative gate potential (−VG) is below threshold voltage (VT).

FIG. 2 schematically shows a solenoid having an electric current inside and positioned behind a double slit in an experiment with a beam of electrons at the same energy level passing through the double slit on opposite sides of the solenoid.

FIG. 3 schematically shows a cross-sectional (XZ) view (A-A) of the transistor of the present invention with ohmic contacts.

FIG. 4 schematically shows the dependence of the source-drain current (a charge carrier density) induced inside the 2DEG channel of a GaN/AlGaN HEMT on the thickness of the AlGaN layer recessed in the open gate area.

FIG. 5 illustrates a theory behind the 2DEG formation (charge neutrality combined with the lowest energy level) at the conduction band discontinuity.

FIG. 6a schematically shows the 2DEG area created in the step of the 2DEG-pattering via ion implantation during the manufacturing process. AZ 4533 is a positive thick resist.

FIG. 6b shows the lithographic mask of the sensor layout of the present invention.

FIG. 6c shows the lithographic image of the 2DEG channel formed with AZ 4533 thick resist lithography over the mask shown in FIG. 5b.

FIGS. 6d-6e show the mask and the corresponding lithographic image, respectively, of the sensor layout of the present invention.

FIG. 6f shows the ±2-μm alignment precision on 25×25 mm2 samples in the lithography of the sensor layout of the present invention.

FIG. 6g shows the lithographic images of the multichannel samples.

FIG. 6h shows the fixed sample on the Si—GaN/AlGaN wafer prepared for ion implantation and containing around 30-32 sensors with 4-8 channels on each sample.

FIG. 6i shows the lithographic image of the sensor layout with the AZ4533 resist after development, prepared for ion implantation.

FIG. 6j shows the 2DEG channels (dark) patterned by ion-implantation after the resist removal.

FIG. 6k shows the visible non-implanted area containing the conductive 2DEG channel.

FIG. 7a shows the AFM surface image of the top recessed layer of the PC-HEMT made by the manufacturing process of the present invention. The measured RMS value of the surface roughness is 0.674 nm in this case.

FIG. 7b shows the AFM surface image of the top recessed layer of the HEMT made by a conventional manufacturing process. The measured RMS value of the surface roughness is 1.211 nm in this case.

FIG. 7c shows the time-dependent plot of the drain-source electric current IDS of the nitrogen oxide sensor of the present invention measuring 100 ppb of the NO₂ gas in humid air, where the sensor is based on the PC-HEMT made by the manufacturing process of the present invention.

FIG. 7d shows the time-dependent plot of the drain-source electric current IDS of the nitrogen oxide sensor measuring 100 ppb of the NO₂ gas in humid air, where the sensor is based on the HEMT made by a conventional manufacturing process.

FIG. 8a schematically shows the formation of the 2DEG and 2DHG channels in the Ga-face three-layer Ga/AlGaN/GaN PC-HEMT structure.

FIG. 8b schematically shows the formation of the 2DEG and 2DHG channels in the N-face three-layer Ga/AlGaN/GaN PC-HEMT structure.

FIG. 9 schematically shows the formation of the 2DEG channel in the N-face three-layer GaN/AlGaN/GaN PC-HEMT structure with an ultrathin AlN or AlGaN layer for improved confinement.

FIG. 10a schematically shows a cross-sectional (XZ) view (A-A) of the transistor of the present invention with ohmic contacts and with the heterojunction structure placed on free-standing membranes.

FIG. 10b illustrates a situation when the external pressure (mass effect) is applied on the sensor incorporating the transistor of FIG. 10a and transferred into a changed internal strain caused by bending.

FIG. 10c schematically shows a cross-sectional (XZ) view (A-A) of the transistor of the present invention with non-ohmic contacts and with the heterojunction structure placed on free-standing membranes.

FIG. 11a shows the metamolecule of the metamaterial of the present invention, supporting toroidal dipolar excitation with incorporated silicon strips. The exemplary diameter of the metamolecule is 30 μm with the central gap equal to 2 μm and the lateral gaps 5 μm. The silicon strips have the gaps 1.4 μm.

FIG. 11b shows the metamolecule as a fragment of the metamaterial of the present invention, supporting toroidal dipolar excitation. Red arrows show displacement currents j induced by the vertically polarized plane wave, blue arrow shows toroidal dipole moments T of the metamolecule, while green arrow shows circulated magnetic moment m (Courtesy of Basharin (2017)).

FIG. 11c shows the amplitude of the conductive currents j induced in the metamolecule at approximately 4.8 THz frequency (Courtesy of Basharin (2017)).

FIG. 11d shows the photographic image of the toroidal metamaterial of the present invention (Courtesy of Basharin (2017)).

FIG. 12a schematically shows the top (XY) view and the basic topology of the transistor of the present invention.

FIGS. 12a-12c schematically show the Configuration 1 basic topology of the transistor of the present invention.

FIGS. 13a-13c schematically show the Configuration 2 basic topology of the transistor of the present invention.

FIGS. 14a-14b schematically show the Configuration 3 basic topology of the transistor of the present invention.

FIGS. 15a-15b schematically show the Configuration 4 basic topology of the transistor of the present invention.

FIGS. 16a-16c schematically show the Configuration 5 basic topology of the transistor of the present invention.

FIG. 17 schematically shows a microelectronic sensor of the present invention for sensing electrical signals in the sub-THz and THz frequency range.

FIG. 18a illustrates the barrier layer/liquid or gas interface with the double layer formation, simplified equivalent interface circuitry and ion electrodynamics during exposure of the sensor to a positive charge.

FIG. 18b illustrates the barrier layer/liquid or gas interface with the double layer formation, simplified equivalent interface circuitry and ion electrodynamics during exposure of the sensor to a negative charge.

FIG. 19a illustrates an equivalent circuit of the PC-HEMT-based sensor of the present invention having the molecular or biomolecular layer immobilised on its surface.

FIG. 19b shows a simplified equivalent circuit of the PC-HEMT-based sensor of the present invention having the molecular or biomolecular layer immobilised on its surface.

FIG. 19c displays the theoretical transistor-transfer function in the most simplified form.

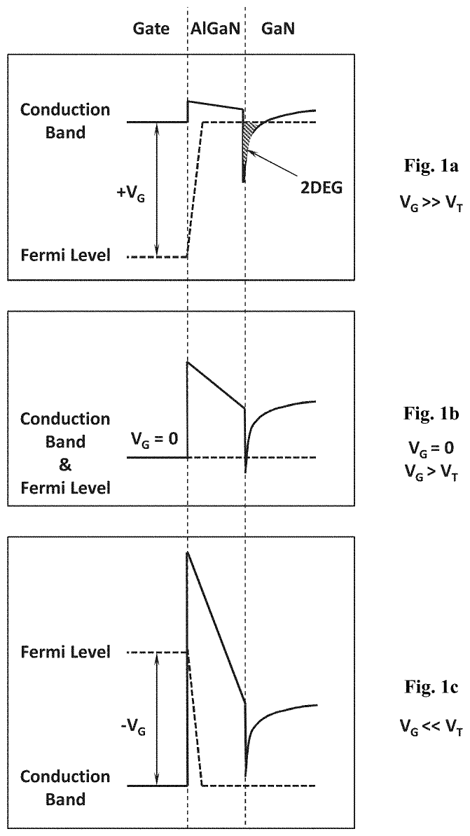

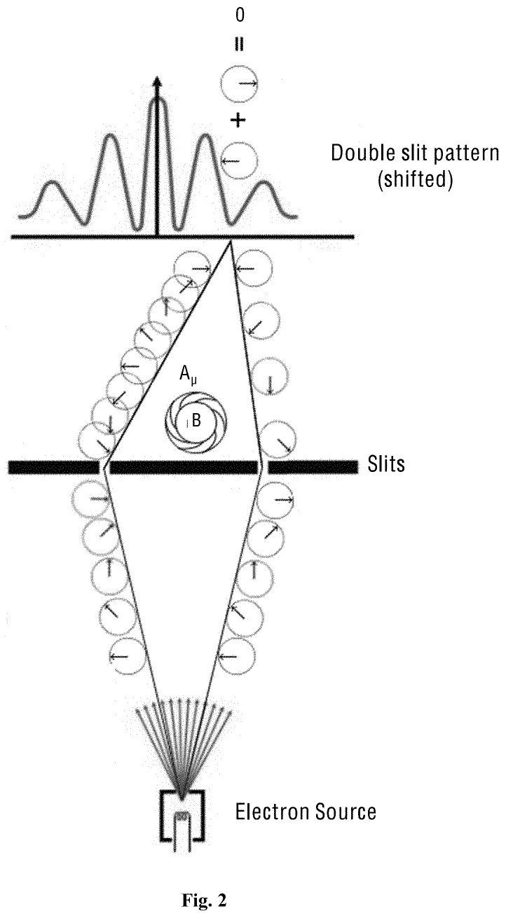

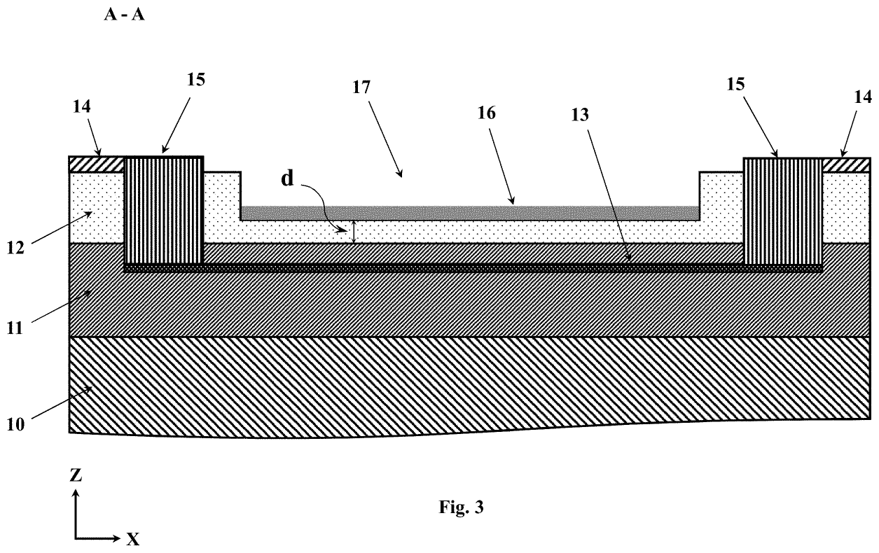

### DETAILED DESCRIPTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising x and z” should not be limited to devices consisting only of components x and z. Also, the scope of the expression “a method comprising the steps x and z” should not be limited to methods consisting only of these steps.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached to”, “connected to”, “coupled with”, “contacting”, etc., another element, it can be directly on, attached to, connected to, coupled with or contacting the other element or intervening elements may also be present. In contrast, when an element is referred to as being, for example, “directly on”, “directly attached to”, “directly connected to”, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Reference is now made to FIG. 3 schematically showing the structure in a cross-sectional (XZ) view (A-A) of the PC-HEMT of the present invention. In one aspect, the present application describes an open-gate pseudo-conductive high-electron mobility transistor (PC-HEMT) comprising:
(i) a multilayer heterojunction structure composed of III-V single-crystalline or polycrystalline semiconductor materials, said structure comprising at least one buffer layer (11) and at least one barrier layer (12), said layers being stacked alternately, and said structure being deposited on a substrate layer (10) or placed on free-standing membranes (18);
(ii) a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current, respectively, in said transistor between source and drain contacts (15);
(iii) the source and drain contacts (15) connected to said 2DEG or 2DHG conducting channel (13) and to electrical metallisations (14) for connecting said transistor to an electric circuit; and
(iv) a metamaterial electrode (16) placed on a top layer between said source and drain contacts (15) in the open gate area (17) of the transistor, said metamaterial electrode (16) is capable of detecting electrical signals in the sub-THz or THz frequency range between 30 GHz to 300 THz;
characterised in that the thickness (d) of the top layer of said heterojunction structure in said open gate area (17) is 5-9 nm which corresponds to the pseudo-conducting current range between normally-on and normally-off operation mode of the transistor, and the surface of said top layer has a roughness of about 0.2 nm or less.

The term “2DEG” mentioned in the present description and claims should not be understood or interpreted as being restricted to the two-dimensional electron gas. As stated above and will be explained later in this application, the two-dimensional hole gas may also be a possible current carrier in a specific heterojunction structure. Therefore, the term “2DEG” may be equally replaced with the term “2DHG” without reference to any particular PC-HEMT configuration.

In a specific embodiment, the III-V semiconductor materials are selected from GaN/AlGaN, GaN/AlN, GaN/InN, GaN/InAlN, InN/InAlN, GaN/InAlGaN, GaAs/AlGaAs and LaAlO₃/SrTiO₃.

The electrical metallisations (14) connect the PC-HEMT to an electric circuit and allow electric current to flow between the source and drain contacts. The electrical metallisations (14) are made of metal stacks, such as Cr/Au, Ti/Au, Ti/W, Cr/Al and Ti/Al. The Cr or Ti layers of the metal stack is, for example, of 5-10 nm thickness, while the second metal layer, such as Au, W and Al, is of 100-400 nm thickness. The actual metallisations (14) are chosen according to the established technology and assembly line at a particular clean room fabrication facility. The source and drain ohmic contacts are usually made of metal stacks, such as Ti/Al/Mo/Au, Ti/Al/Ni/Au, Ti/Au and Ti/W having the thickness of 15-50 nm. The non-ohmic contacts on the other hand are capacitively coupled to the conducting 2DEG channel (13) via displacement currents (25).

In yet further embodiment, substrate layer (10) comprises a suitable material for forming the barrier layer and is composed, for example, of sapphire, silicon, silicon carbide, gallium nitride or aluminium nitride. The hetero junction structure (11, 12) is deposited on the substrate layer (10), for example, by a method of metalorganic chemical vapour deposition (MOCVD), and forms a two-dimensional electron gas (2DEG) channel (13) in the close proximity to the interface between the buffer layer (11) and the top barrier layer (12). The top barrier layer (12) is then either recessed or grown as a thin layer between the source and drain contacts, thereby forming an open gate area.

The 2DEG/2DHG channel (13) formed near the interface between the buffer layer (11) and the barrier layer (12) serves as a main sensitive element of the transistor reacting to a surface charge and potential. The 2DEG/2DHG channel (13) is configured to interact with very small variations in surface or proximal charge or changes of electrical field on the barrier layer/liquid-air or barrier layer/metal/liquid-air interfaces interacting with the donor-like surface trap states of the barrier layer. This will be defined and discussed below in detail.

“Open gate area” (17) of the PC-HEMT is defined as an area between the source and drain contacts (15) of the transistor which is directly exposed to a conductive medium, such as liquid or gas capable of conducting current. An example of the conductive liquid is an electrolyte saline solution. In this case, instead of the fixed gate voltage, which is normally applied to a gate electrode, a reference potential is applied to the electrolyte-semiconductor system, via an optional reference electrode that is dipped into the electrolyte. As a result, in the absence of the physical gate, the electrolyte itself becomes an open gate of the transistor. This will be explained in more detail below.

The specific thickness of the top barrier layer (12) in the open gate area is achieved by either dry etching the semiconductor material of the barrier layer (12), i.e. recessing the layer in the open gate area with the etching rate of 1 nm per 1-2 min in a controllable process, or coating the buffer layer (11) in the open gate area with an ultrathin layer of the III-V semiconductor material. In order to increase the charge sensitivity of the transistor, the surface of the recessed ultrathin barrier layer is post-treated with plasma (chloride) epi-etch process. Consequently, the natively passivated surface is activated by the plasma etch to create an uncompensated (ionised) surface energy bonds or states, which are neutralized after MOCVD growing.

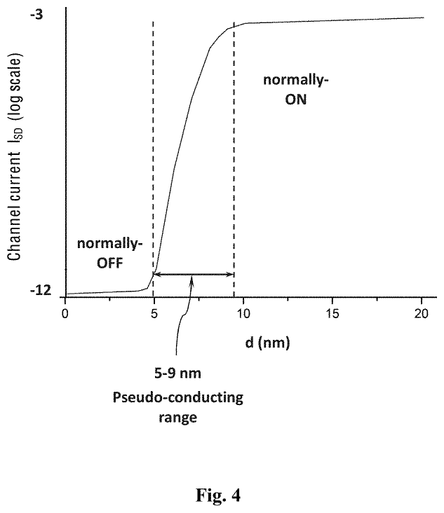

FIG. 4 shows the dependence of the source-drain current (a charge carrier density) on the barrier layer thickness recessed in the open gate area. As seen from the plot, the HEMTs that have a thickness of the barrier layer in the open gate area larger than about 9 nm are normally-on devices. In such devices, due to the inherent polarisation effects present in the III-V materials, a thin sheet of charges is induced at the top and bottom of the interfaces of the barrier layer. As a result, a high electric field is induced in the barrier layer, and surface donor states at the top interface start donating electrons to form the 2DEG channel at the proximity of the hetero junction interface without the application of a gate bias. These HEMTs are therefore normally-on devices. On the other hand, the HEMTs that have a thickness of the barrier layer in the open gate area lower than about 5 nm act as normally-off devices.

The top barrier layer recessed or grown in the open gate area to 5-9 nm is optimised for significantly enhancing sensitivity of the PC-HEMT sensor. This specific thickness of the top barrier layer in the open gate area corresponds to the “pseudo-conducting” current range between normally-on and normally-off operation modes of the transistor and requires further explanation.

“Pseudo-conducting” current range of the HEMT is defined as an operation range of the HEMT between its normally-on and normally-off operation modes. “Trap states” are states in the band-gap of a semiconductor which trap a carrier until it recombines. “Surface states” are states caused by surface reconstruction of the local crystal due to surface tension caused by some crystal defects, dislocations, or the presence of impurities. Such surface reconstruction often creates “surface trap states” corresponding to a surface recombination velocity. Classification of the surface trap states depends on the relative position of their energy level inside the band gap. The surface trap states with energy above the Fermi level are acceptor-like, attaining negative charge when occupied. However, the surface trap states with energy below the Fermi level are donor-like, positively charged when empty and neutral when occupied. These donor-like surface trap states are considered to be the source of electrons in the formation of the 2DEG channel They may possess a wide distribution of ionization energies within the band gap and are caused by redox reactions, dangling bonds and vacancies in the surface layer. A balance always exists between the 2DEG channel density and the number of ionised surface donors which is governed by charge neutrality and continuity of the electric field at the interfaces.

Thus, the donor-like surface traps formed at the surface of the barrier layer of the HEMT are one of the most important sources of the 2DEG in the channel. However, this only applies for a specific barrier layer thickness. In a relatively thin top barrier layer, the surface trap state is below the Fermi level. However, as the top barrier layer thickness increases, the energy of the surface trap state approaches the Fermi energy until it coincides with it. The thickness of the top barrier layer corresponding to such situation is defined as “critical”. At this point, electrons filling the surface trap state become pulled to the channel by the strong polarisation-induced electric field found in the barrier to form the 2DEG instantly.

If the surface trap states are completely depleted, further increase in the barrier layer thickness will not increase the 2DEG density. Actually, if the 2DEG channel layer fails to stretch the barrier layer, the later will simply relax. Upon relaxation of the barrier layer, crystal defects are created at the interface between the buffer layer and the barrier layer, and the piezoelectric polarisation instantly disappears causing deterioration in the 2DEG density.

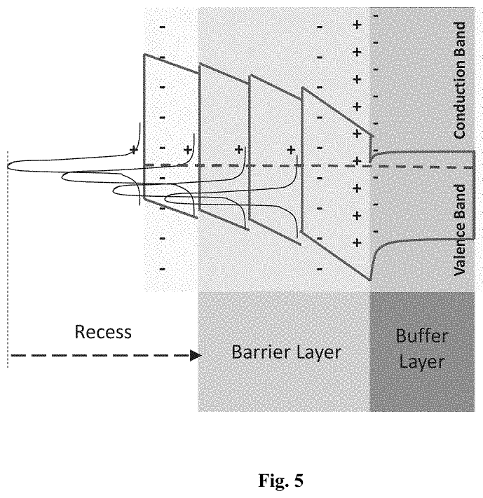

In order to illustrate the above phenomenon of pseudo-conducting current, reference is now made to the following figures. As mentioned above, FIG. 4 shows the dependence of the source-drain current (a charge carrier density) on the recessed top layer thickness. An energy equilibrium between the donor surface trap states and the top layer tunnel barrier leads to the 2DEG formation (charge neutrality combined with the lowest energy level) at the conduction band discontinuity. As explained above, decrease in the thickness of the top barrier layer results in increase of the energy barrier. As a result, the ionisable donor-like surface trap states, which are responsible for electron tunnelling from the surface to 2DEG, drift bellow the Fermi level, thereby minimizing the electron supply to the 2DEG channel. This theoretical situation is schematically shown illustrated in FIG. 5. Therefore, the recess of the top layer from 9 nm to 5 nm leads to extremely huge drop in the 2DEG conductivity for six orders of magnitude.

In view of the above, it is clear that the mechanism of the 2DEG depletion based on recessing the top barrier layer is strongly dependent on the donor-like surface trap states (or total surface charge). As the thickness of the barrier layer decreases, less additional external charge is needed to apply to the barrier layer surface in order to deplete the 2DEG channel There is a critical (smallest) barrier thickness, when the conducting 2DEG channel is mostly depleted but still highly conductive due to a combination of the energy barrier and the donor surface trap states energy. At this critical thickness, even the smallest energy shift at the surface via any external influence, such as surface reaction, charging etc., leads immediately to very strong 2DEG depletion. As a result, the surface of the top barrier layer at this critical thickness is extremely sensitive to any smallest change in the electrical field of the surroundings.

Thus, it has been found that the recess of the top layer in the open gate area from 9 nm down to 5 nm drastically reduces the 2DEG density, brings the transistor to the “near threshold” operation and results in highly increased surface charge sensitivity. The specific 5-9 nm thickness of the transistor's top layer responsible for its surprising pseudo-conducting behaviour gives the transistor the incredible sensitivity. So, when it comes into a contact with an ionic fluid or body skin, it opens up the gate to be able to do the ultrasensitive sensing. This thickness must be optimised for significantly enhancing sensitivity of the sensor. This specific thickness of the top layer was surprisingly found to correspond to the “pseudo-conducting” current range between normally-on and normally-off operation modes of the 2DEG channel and requires further explanation.

The top layer is recessed to this specific thickness after subjecting to short plasma activation by an ultra-low damage reactive-ion etching technique using inductively-coupled plasma (ICP) with a narrow plasma-ion energy distribution. Such short plasma treatment allows much lower roughness of the surface, which is a function of the semiconductor vertical damage depth during the plasma etching process. Such low surface roughness (about 0.2 nm and less) can be achieved only via this ICP-RIE ultra low damage etching process with a narrow plasma-ion energy distribution, and this inherently results in a very low vertical damage depth to the top layer, which allows the minimal surface scattering and minimal surface states-2DEG channel interaction with the maximum signal-to-noise ratio of the sensor. Thus, the depth effect of the vertical sub-nanometre damage to the top recessed layer, due to an ultra-low damage ICP-RIE etching process with a very narrow plasma-ion energy distribution, is the only way to optimally achieve the required sub-nanometre roughness of the semiconductor surface. This inherently results in an adjustable pseudo-conductive working point with the highest charge sensitivity ever possible. This depth effect is always inherent to the sub-nanometre roughness of the semiconductor surface, which was measured using AFM (atomic force microscope).

Thus, in addition to the recessed top layer thickness, roughness of the top layer surface is another very important parameter that has not been previously disclosed. It has been surprisingly found that the roughness of the top layer surface (in the open gate sensitive area) bellow 0.2 nm prevents scattering of the donor-like surface trap states. Thus, combination of these two features: 5-9 nm thickness of the top layer in the open gate area and strongly reduced roughness of its surface (bellow 0.2 nm) make the sensor incredibly sensitive.

In a certain aspect, the method for manufacturing of the PC-HEMTs of the present invention comprises the following steps:
Step 1: Plasma-enhanced atomic layer deposition (ALD) of alumina (Al₂O₃) on a pre-aligned masked Si—GaN/AlGaN wafer with nitrogen-plasma de-trapping for the thickness of the Al₂O₃ layer being 3-10 nm. The Al₂O₃ layer thickness was measured with an X-ray reflectometer.
Step 2: Plasma-enhanced atomic layer deposition (ALD) pattering of the wafer coated with the thin Al₂O₃ layer in Step 1, with hydrogen fluoride (HF) or using the aforementioned reactive-ion etching (RIE) technique.
Step 3: Optionally creating the source and drain ohmic contacts (in case ohmic contacts are required) on the coated wafer obtained in Step 2 from metal stacks, for example Ti/Al/Mo/Au, Ti/Al/Ni/Au, Ti/Au and Ti/W, having 15-50 nm thickness, using spin-coating technique or e-beam physical vapour deposition (VPD) of the stack metals. The deposition rates using the e-VPD technique were determined for the ohmic-stack metals using the Dektak Profilometer with dummy lift-off samples.
Step 4: Two-dimensional electron gas (2DEG) channel-pattering of the wafer obtained in Step 3 with argon- or nitrogen-ion implantation.
Step 5: Plasma-enhanced chemical vapour deposition (CVD) of the ONO stack over the wafer obtained in Step 4. This is the stress-free technique to deposit the layer of the SiO—SiN—SiO stack having an exemplary thickness of about 200-300 nm and structured by the ICP-RIE dry etching, which is the CF4-based etching method. In this step, the pseudo-conducting channel areas and ohmic electrical contact pads of the transistor become available.
Step 6: Optional lift-off deposition of an Au or Ti/W-CMOS-gate electrode (in case a gate electrode is to be deposited on the top layer of the heterojunction structure for an integrated MMIC-HEMT-based amplifier manufacturing).
Step 7: Optional plasma-enhanced ALD pattering with RIE or HF above sensing area (in case the plasma-enhanced ALD layer deposited in Step 1 is removed separately to ONO stack).
Step 8: Atomic layer etching (ALE) of the wafer obtained in Steps 5-7. This sophisticated technique carried out in the clean manufacturing cluster of the applicant is the only technique allowing the removal of individual atomic layers (the top atomic layers of the wafer). ALE is a way better-controlled technique than RIE, though it has not been commercially used until now because very sophisticated gas handling is required, and removal rates of one atomic layer per second are the real state of the art. This step is the step of creating the pseudo-conducting working point of the transistor, because ALE allows achieving the specific thickness of 5-9 nm thickness of the top layer in the open gate area with the extremely low surface roughness of the top layer below 0.2 nm.
Step 9: Optional plasma-enhanced CVD or ALD of the dielectric layer used for device passivation and in some gas sensors.
Step 10: Optional deep reactive-ion etching (DRIE or Bosch process) of the Si-substrate under sensing areas (in case the substrate is on the free-standing membranes—used, for example, in RF-HEMTs, FBAR and SAW sensors).

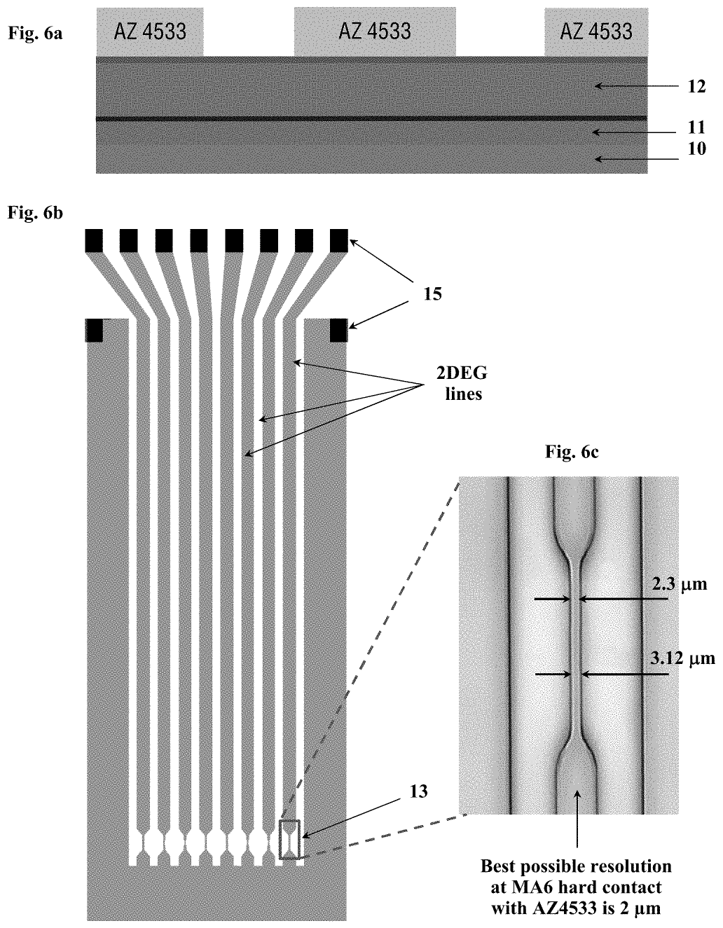

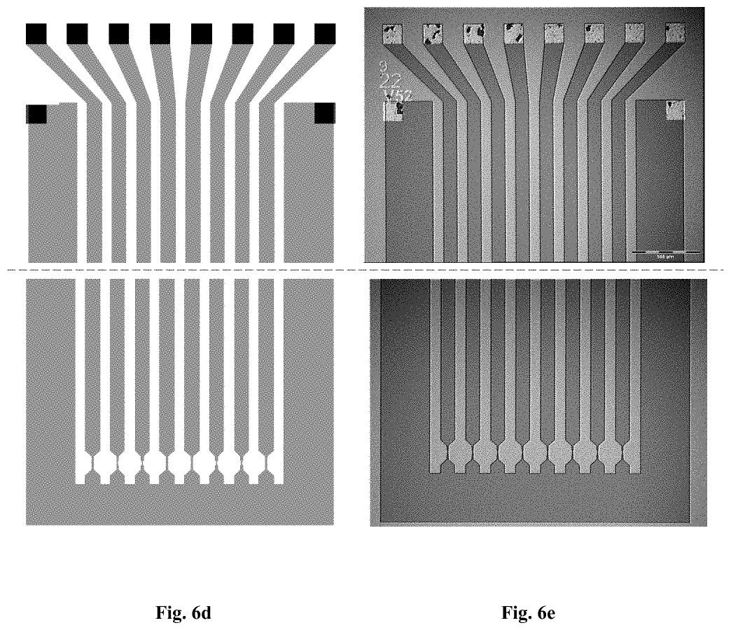

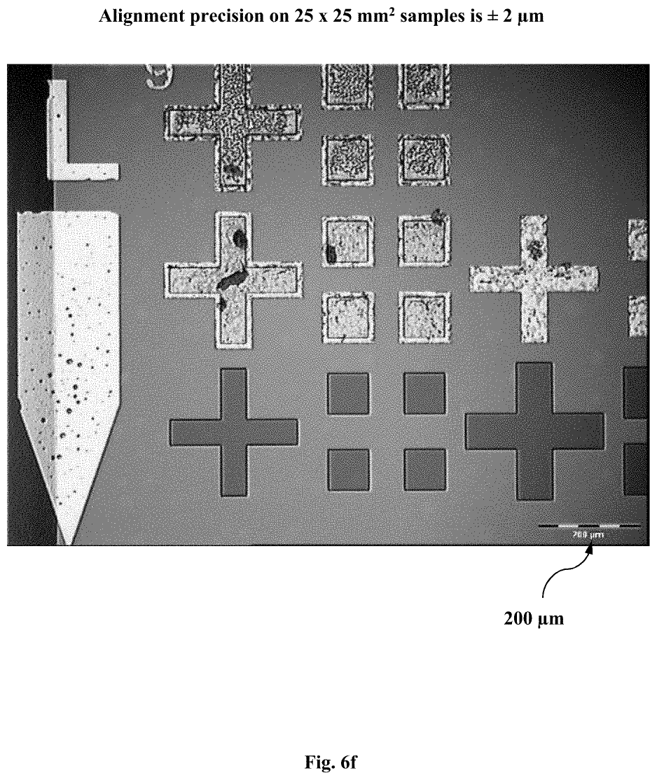

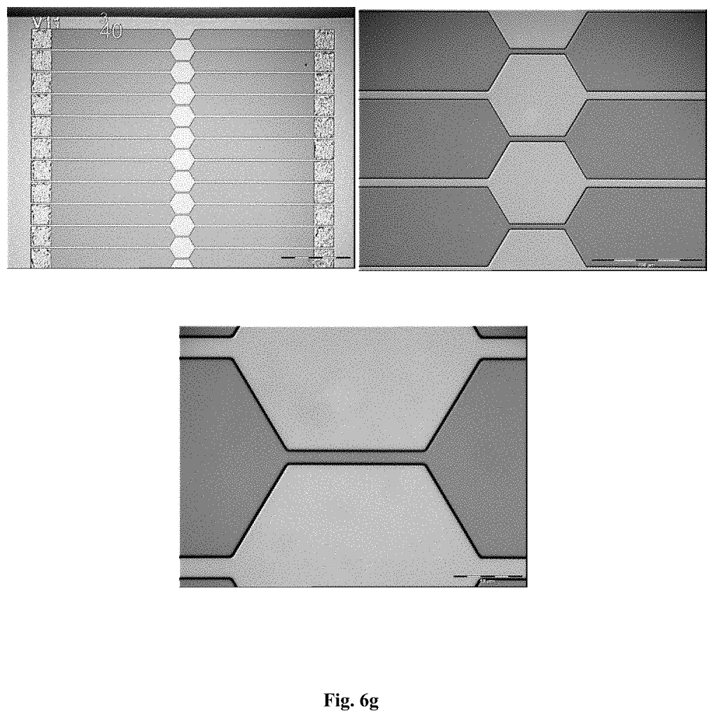

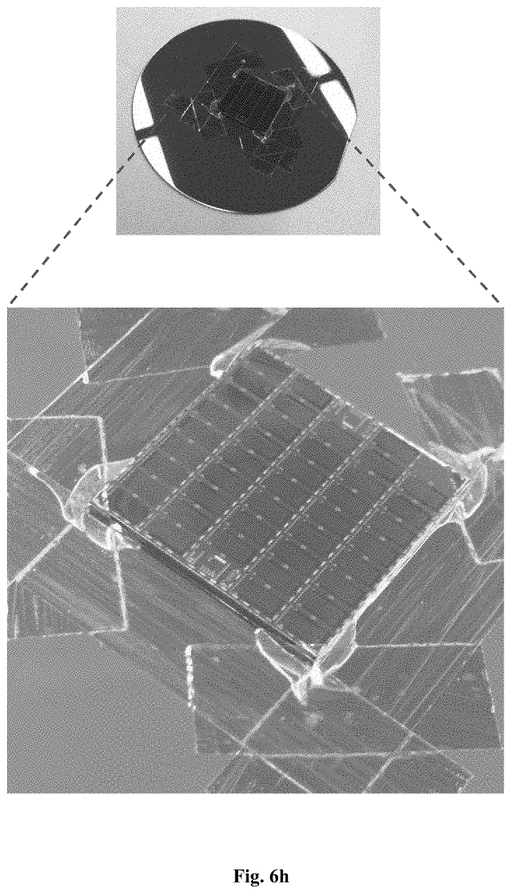

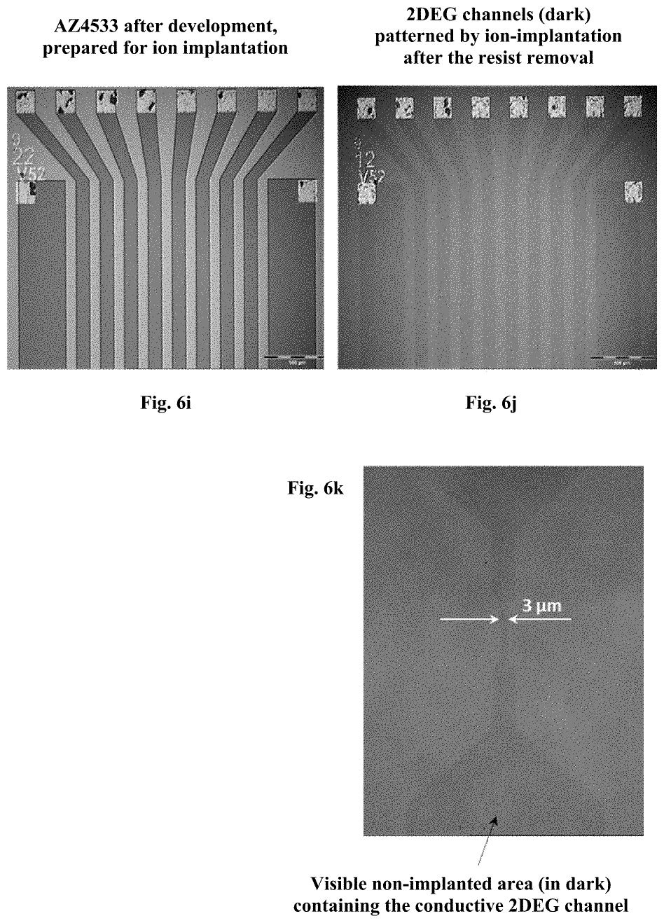

Reference is now made to FIGS. 6a-6c showing the sensor, which is obtained in Step 4 of the 2DEG-channel pattering. The lithography of the sensor was performed with AZ 4533, which is a positive thick resist having optimised adhesion for common wet etching. The lithographic resist film thickness obtained at 7000-rpm spin speed and at 100° C. for 1 min was 3 μm. Thus, as seen in the lithographic image of FIG. 6c, the formed 2DEG channel (13) is approximately 2-3 μm wide. The overall exposure time was 9 sec, followed by 5-min development in MIF726 developer.

FIG. 6d-6e show the mask and corresponding lithographic image, respectively, of the sensor layout of the present invention. FIG. 6f demonstrates the high alignment precision of ±2-μm on 25×25 mm² samples in the lithography of the sensor layout of the present invention. FIG. 6g shows the lithographic images of the multichannel samples. FIG. 6h shows the fixed sensor chip sample on the Si—GaN/AlGaN wafer, which contains approximately 30-32 sensors with 4-8 channels on each sample and prepared for ion implantation. FIG. 6i shows the obtained lithographic image of the present sensor layout with the AZ4533 resist after development, prepared for ion implantation. FIG. 6j shows the 2DEG channels (dark) patterned by ion-implantation after the resist removal. The argon-ion implantation was conducted with 20 keV and 30 keV energies and with an exemplary dose of 2.5e¹³/cm² and a 7° tilt angle. AZ4533 was removed with oxygen plasma at 220 W for 10 min. FIG. 6k shows the visible non-implanted area containing the conductive 2DEG channel.

The atomic layer etching (ALE) performed in Step 8 of the manufacturing process is the most important stage in the process. As mentioned above, it allows the controlled recess of a top layer, removing a single atomic layer-by-layer, where the etch thickness is in the order of magnitude of a single atomic monolayer. As explained above, such ultra-low damage to the top layer of the heterogeneous structure, when the actual surface roughness is controlled by a single atomic monolayer, allows to achieve the sub-nanometre roughness (about 0.2 nm and less) of the top layer when its thickness is only few nanometres (5-9 nm).

The ALE process sequence consists of repeated cycling of process conditions. The total amount of material removed is determined by the number of repeated cycles. Each cycle is typically comprised of four steps: adsorption, first purge, desorption and second purge. During the adsorption step of the cycle, reactive species are generated in the reactor (for example, upon plasma excitation), adsorbed by, and react with material on the wafer. Due to the self-limiting process, and with the proper choice of reactants and process conditions, reaction takes place with only a thin layer of material, and the reaction by-products are formed. This step is followed by purging of the reactor to remove all traces of the reactant. Then the by-product desorption takes place due to bombardment of the wafer surface by noble gas ions with a tightly controlled energy. Again, by-products are purged from the reactor, and the wafer is ready for the last two (optional) steps of the manufacturing process.

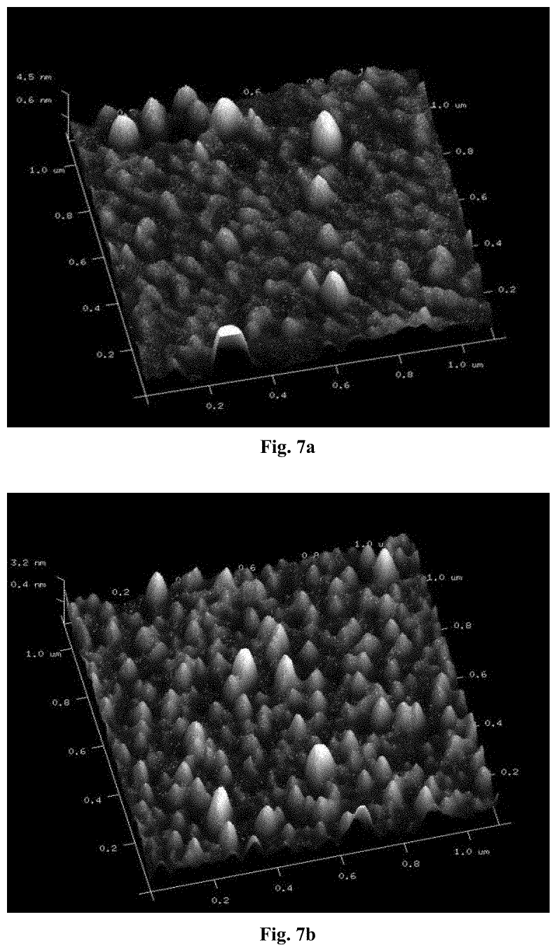

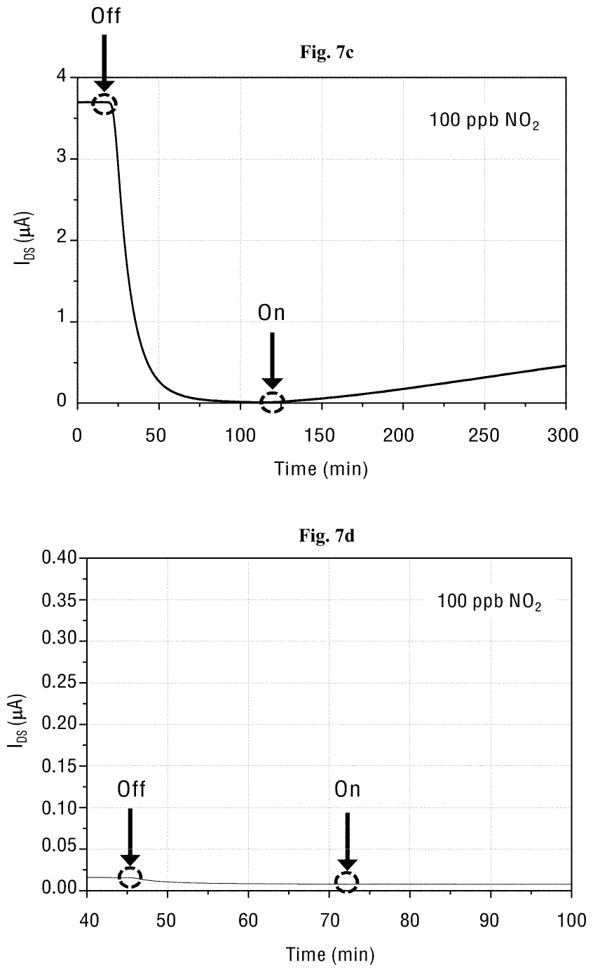

Reference is now made to FIG. 7a showing the AFM image of the top recessed layer surface of the PC-HEMT produced by the manufacturing process of the present invention. The measured RMS value of the surface roughness is 0.674 nm in this case. FIG. 7b shows the AFM surface image of the top recessed layer of the HEMT made by a conventional manufacturing process. In this conventional process, the HEMT initially had a top ultrathin-grown AlGaN layer of the 6-7 nm thickness. This layer was recessed with inductively-coupled plasma (ICP) for 60 sec using a conventional reactive-ion etching (RIE) technique. The measured RMS value of the surface roughness is 1.211 nm in this case. FIG. 7c show the time-dependent plot of the drain-source electric current IDS of the nitrogen oxide sensor measuring 100 ppb of the NO₂ gas in 80%-humid air, where the sensor incorporates the PC-HEMT made by the manufacturing process of the present invention. FIG. 7d show the time-dependent plot of the IDS of the nitrogen oxide sensor measuring 100 ppb of the NO₂ gas in 80%-humid air, where the sensor incorporates and based on the HEMT made by the conventional manufacturing process. It is clear from these comparative examples that the manufacturing process of the present invention based on the ultra-low damaging RIE with a narrow plasma-ion energy distribution leads to much lower roughness of the semiconductor surface, which in turn leads to incredibly high sensitivity of the sensor.

Thus, the significant features of the PC-HEMT of the present invention are:
(i) the thickness of the top layer in the open gate area is 5-9 nm, preferably 6-7 nm, more preferably 6.3 nm, which corresponds to the pseudo-conducting current range between normally-on and normally-off operation mode of the transistor,
(ii) the surface of the top layer has a roughness of 0.2 nm or less, preferably 0.1 nm or less, more preferably 0.05 nm, and
(iii) a metamaterial electrode placed on a top layer between the source and drain contacts in the open gate area of the transistor (this will be discussed below).

In a further aspect, the hetero junction structure is a three-layer structure consisting of two GaN layers and one AlGaN layer squeezed between said GaN layers like in a sandwich, wherein the top layer is a GaN layer. This may lead to formation of the two-dimensional hole gas (2DHG) in the top GaN layer above the AlGaN layer which results in reversing polarity of the transistor compared to the two-layer structure discussed above.

In general, polarity of III-V nitride semiconductor materials strongly affects the performance of the transistors based on these semiconductors. The quality of the wurtzite GaN materials can be varied by their polarity, because both the incorporation of impurities and the formation of defects are related to the growth mechanism, which in turn depends on surface polarity. The occurrence of the 2DEG/2DHG and the optical properties of the hetero junction structures of nitride-based materials are influenced by the internal field effects caused by spontaneous and piezoelectric polarizations. Devices in all of the III-V nitride materials are fabricated on polar {0001} surfaces. Consequently, their characteristics depend on whether the GaN layers exhibit Ga-face positive polarity or N-face negative polarity. In other words, as a result of the wurtzite GaN materials polarity, any GaN layer has two surfaces with different polarities, a Ga-polar surface and an N-polar surface. A Ga-polar surface is defined herein as a surface terminating on a layer of Ga atoms, each of which has one unoccupied bond normal to the surface. Each surface Ga atom is bonded to three N atoms in the direction away from the surface. In contrast, an N-polar surface is defined as a surface terminating on a layer of N atoms, each of which has one unoccupied bond normal to the surface. Each surface N atom is also bonded to three Ga atoms in the direction away from the surface. Thus, the N-face polarity structures have the reverse polarity to the Ga-face polarity structures.

As described above for the two-layer heterojunction structure, the barrier layer is always placed on top of the buffer layer. The layer which is therefore recessed in the two-layer heterojunction structure is the barrier layer, specifically the AlGaN layer. As a result, since the 2DEG is used as the conducting channel and this conducting channel is located slightly below the barrier layer (in a thicker region of the GaN buffer layer), the hetero junction structure is grown along the {0001}-direction or, in other words, with the Ga-face polarity. However, as explained above, the physical mechanism that leads to the formation of the 2DEG is a polarisation discontinuity at the AlGaN/GaN interface, reflected by the formation of the polarisation-induced fixed interface charges that attract free carriers to form a two-dimensional carrier gas. It is a positive polarisation charge at the AlGaN/GaN interface that attracts electrons to form 2DEG in the GaN layer slightly below this interface.

As noted above, polarity of the interface charges depends on the crystal lattice orientation of the hetero junction structure, i.e. Ga-face versus N-face polarity, and the position of the respective AlGaN/GaN interface in the hetero junction structure (above or below the interface). Therefore, different types of the accumulated carriers can be present in the hetero junction structure of the embodiments.

In case of the three-layer hetero junction structure, there are four possible configurations:

### Ga-Face Polarity

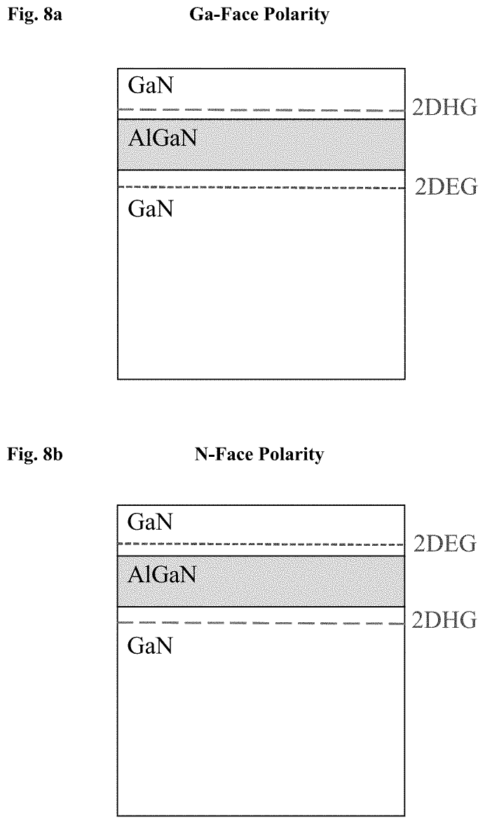

1) The Ga-face polarity is characterised by the 2DEG formation in the GaN layer below the AlGaN barrier layer. This is actually the same two-layer configuration as described above, but with addition of the top GaN layer. In this configuration, the AlGaN barrier layer and two GaN layers must be nominally undoped or n-type doped.
2) In another Ga-face configuration shown in FIG. 8a, in order to form the conducting channel comprising a two-dimensional hole gas (2DHG) in the top GaN layer above the AlGaN barrier layer in the configuration, the AlGaN barrier layer should be p-type doped (for example, with Mg or Be as an acceptor) and the GaN buffer layer should be also p-type doped with Mg, Be or intrinsic.

### N-Face Polarity

3) The N-face polarity is characterised by the 2DEG formation in the top GaN layer above the AlGaN barrier layer, as shown in FIG. 8b. In this case, the AlGaN barrier layer and two GaN buffer layers must be nominally undoped or n-type doped.
4) The last configuration assumes that the 2DHG conducting channel is formed in the buffer GaN layer below the AlGaN barrier layer. The top GaN layer may be present (three-layer structure) or not (two-layer structure) in this case. The AlGaN barrier layer must be p-type doped (for example, with Mg or Be as an acceptor) and the bottom GaN layer should be also p-type doped with Mg, Be or intrinsic.

Thus, there are four hetero junction three-layer structures implemented in the transistor of the embodiments, based on the above configurations:
A. Ga-Face GaN/AlGaN/GaN heterostructure with the 2DEG formed in the GaN buffer layer below the AlGaN barrier layer. In this case, the top GaN layer may be omitted to obtain the two-layer structure. For the three-layer structure, the top GaN layer must be recessed to 1-9 nm thickness in the open gate area or grown with this low thickness, with the roughness below 0.2 nm, and the thickness of the AlGaN barrier can be adjusted properly during growth.
B. Ga-Face GaN/AlGaN/GaN heterostructure with the 2DHG conducting channel formed in the top GaN layer above the AlGaN barrier layer. The top GaN layer must be recessed to 5-9 nm thickness in the open gate area with the roughness below 0.2 nm, and the thickness of the AlGaN barrier layer can be adjusted properly. P-type doping concentrations of the GaN layer and AlGaN barrier have to be adjusted; the 2DHG has to be contacted (in the ideal case by ohmic contacts).
C. N-Face GaN/AlGaN/GaN heterostructure with the 2DEG in the top GaN layer above the AlGaN barrier layer. The top GaN layer must be recessed to 5-9 nm thickness in the open gate area with the roughness below 0.2 nm. Thickness of the AlGaN barrier can be adjusted during growth. N-type doping levels of the GaN buffer layer and the AlGaN barrier layer must be adjusted; the 2DEG has to be contacted (in the ideal case by ohmic contacts).
D. N-Face GaN/AlGaN/GaN heterostructure with the 2DHG in the GaN buffer layer below the AlGaN barrier layer. In this case, the top GaN layer may be omitted to obtain the two-layer structure. In both, the two-layer and three-layer configurations, the top GaN layer must be recessed to 1-9 nm thickness in the open gate area with the roughness below 0.2 nm, and the thickness of the AlGaN barrier can be adjusted properly.

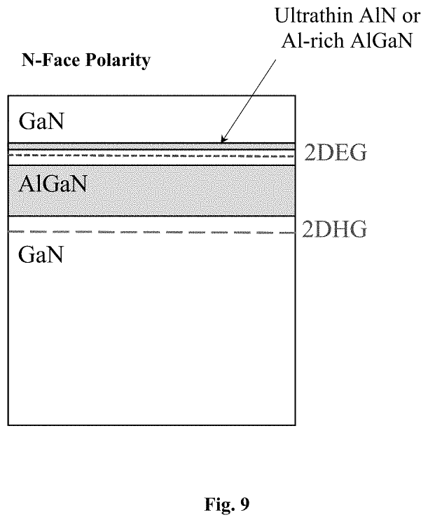

In all the above structures, the deposition of a dielectric layer on top might be beneficial or even necessary to obtain a better confinement (as in case of the N-face structures). As shown in FIG. 9, for the above “C” structure, it may be even more beneficial to include an ultrathin (about 1 nm) AlN or AlGaN barrier layer with high Al-content on top of the 2DEG channel to improve the confinement.

The preferable structures of the embodiments are structures “B”, “C” and “D”. In the structure “B”, the 2DHG conducting channel formed in the top GaN layer, which has a higher chemical stability (particularly towards surface oxidation) than the AlGaN layer. Concerning the structure “C”, the 2DEG conducting channel might be closer to the surface. Therefore, the electron mobility might be lower than in the 2DEG structure with the Ga-face polarity. In general, the polarity of the heterostructure can be adjusted by the choice of the substrate (e.g. C-face SiC) or by the growth conditions.

Based on the above, one of the aspects of the present application is the open-gate pseudo-conductive high-electron mobility transistor (PC-HEMT) comprising:
(i) a multilayer hetero junction structure made of gallium nitride (GaN) and aluminium gallium nitride (AlGaN) single-crystalline or polycrystalline semi-conductor materials, deposited on a substrate layer or placed on free-standing membranes, and characterised in that:
   (a) said structure comprises (i) one top AlGaN layer recessed in an open gate area of the transistor to the thickness of 5-9 nm and having the surface roughness of 0.2 nm or less, and (ii) one bottom GaN buffer layer; said layers have Ga-face polarity, thus forming the two-dimensional electron gas (2DEG) conducting channel in said GaN layer, close to the interface with said AlGaN layer; or
   (b) said structure comprises (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have Ga-face polarity, thus forming a two-dimensional hole gas (2DHG) conducting channel in the top GaN layer, close to the interface with said AlGaN barrier layer; or
   (c) said structure comprises (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have N-face polarity, thus forming a two-dimensional electron gas (2DEG) conducting channel in the top GaN layer, close to the interface with said AlGaN barrier layer; or
   (d) said structure comprises (i) one top AlGaN layer recessed in an open gate area of the transistor to the thickness of 5-9 nm and having the surface roughness of 0.2 nm or less, and (ii) one bottom GaN buffer layer; said layers have N-face polarity, thus forming a two-dimensional hole gas (2DHG) conducting channel in the GaN buffer layer, close to the interface with said AlGaN barrier layer;
(ii) source and drain contacts connected to said 2DEG or 2DHG conducting channel and to electrical metallisations for connecting said transistor to an electric circuit; and
(iii) a metamaterial electrode placed on the top layer between said source and drain contacts in the open gate area of the transistor, said electrode is capable of detecting electrical signals in the frequency range between 30 GHz to 300 THz.

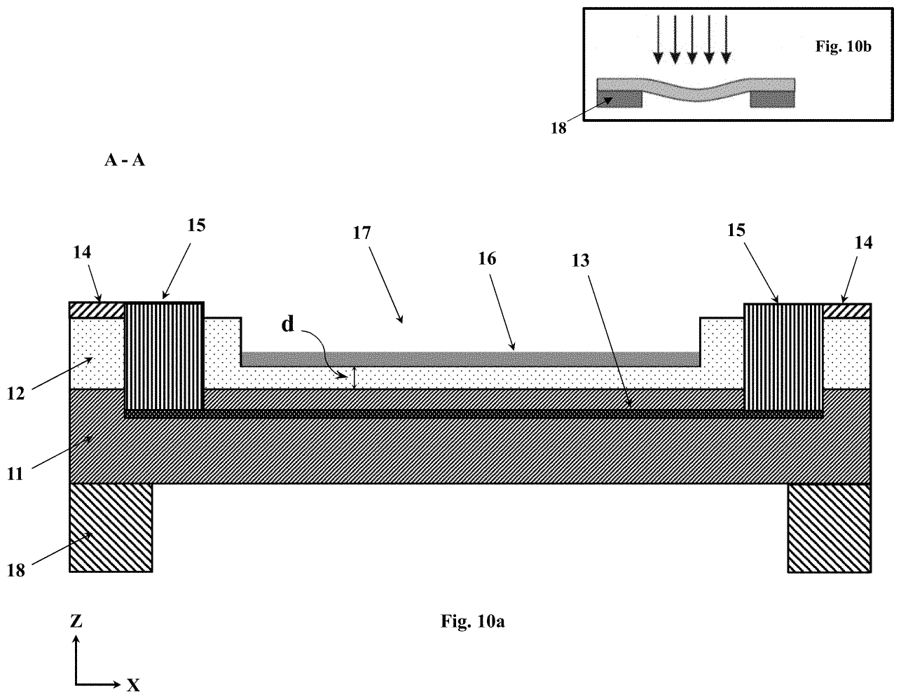

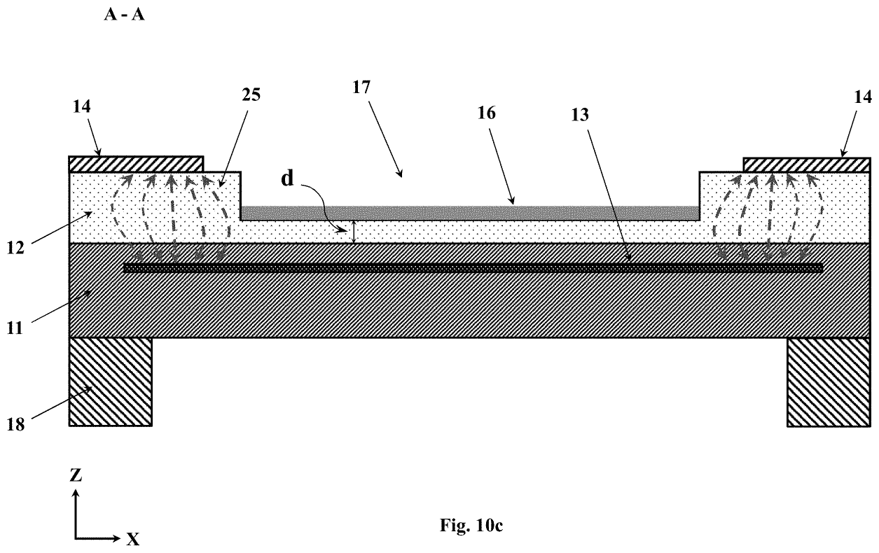

FIG. 10a shows a cross-sectional view of the PC-HEMT configuration of an embodiment with free-standing membranes, comprising:
(i) a multilayer heterojunction structure composed of III-V single-crystalline or polycrystalline semiconductor materials, said structure comprising at least one buffer layer (11) and at least one barrier layer (12), said layers being stacked alternately, and said structure being placed on free-standing membranes (18);
(ii) a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current, respectively, in said transistor between source and drain contacts (15);
(iii) the source and drain contacts (15) connected to said 2DEG or 2DHG conducting channel (13) and to electrical metallisations (14) for connecting said transistor to an electric circuit; and
(iv) a toroidal metamaterial electrode (16) placed on a top layer between said source and drain contacts (15) in the open gate area (17) of the transistor, said toroidal metamaterial electrode (16) is capable of detecting signals in the frequency range between 30 GHz to 300 THz;
characterised in that the thickness (d) of the top layer of said heterojunction structure in said open gate area (17) is 5-9 nm which corresponds to the pseudo-conducting current range between normally-on and normally-off operation mode of the transistor, and the surface of said top layer has a roughness of about 0.2 nm or less.

The PC-HEMT shown in FIG. 10a with the free-standing membranes may be used in “pressure-sensitive” sensors of an embodiment, which are capable of measuring very small pressures. These sensors use the free-standing membranes for creating a mass-loading effect which makes it possible to increase selectivity of the sensors via adding mechanical stress (mass-loading effect) as an additional parameter of the PC-HEMT-based sensor. The free-standing membranes (18) are very flexible free-standing columns of substrate composed of sapphire, silicon, silicon carbide, gallium nitride or aluminium nitride, preferably gallium nitride, having thickness of 0.5-2 μm. The free-standing substrate membranes are very sensitive to any tensile, compressive or mechanical stress changes on the surface of the multilayer hetero junction structure. This results in a mass loading effect, which will be discussed below.

In general, mechanical sensors, much like pressure sensors, are based on the measurement of the externally induced strain in the heterostructures. The pyroelectric properties of group-III-nitrides, such as gallium nitride (GaN), allow two mechanisms for strain transduction: piezoelectric and piezoresistive. The direct piezoelectric effect is used for dynamical pressure sensing. For measurements of static pressure, such sensors are not suitable due to some leakage of electric charges under the constant conditions. For static operation, the piezoresistive transduction is more preferable.

Piezoresistive sensors using wide band gap materials have been previously employed using hexagonal silicon carbide bulk materials for high temperature operation. The piezoresistivity of GaN and AlGaN structures was found to be comparable to silicon carbide. However, piezoresistivity can be further amplified by HEMT structure, as taught by Martin Eickhoff et al in “Piezoresistivity of AlₓGa₁₋ₓN layers and AlₓGa₁₋ₓN/GaN heterostructures”, Journal of Applied Physics 90, 2001, 3383.

For piezoresistive strain sensing at relatively lower pressures (or pressure differences), diaphragm or membranes should be used, where the external pressure is transferred into a changed internal strain caused by bending, as shown in FIG. 10b. The resulting change in polarization alters the 2DEG channel current which is measured.

Eickhoff et al (2001) conducted the first experiments on AlGaN/GaN hetero-structures where the 2DEG channel confined between the upper GaN and AlGaN barrier layer and demonstrated the linear dependence of the 2DEG channel resistivity on the applied strain. Moreover, a direct comparison to cubic SiC and a single AlGaN layer clearly demonstrated the superior piezoresistive properties of the latter. From these results, it is clear that the interaction of piezoelectric and piezoresistive properties improves the sensitivity of pressure sensors by using GaN/AlGaN heterostructures confined with the 2DEG channel.

Thus, the sensor configuration schematically shown in FIGS. 10a-10c involves piezoelectrically coupled, charge and mass sensitive, free-standing GaN membranes, which are prepared, for example, according to U.S. Pat. No. 8,313,968, and offer an elegant and effective solution to achieve both downscaling and an integrated all-electrical low-power sensing-actuation. As mentioned above, GaN exhibits both, piezo- and pyro-electrical properties, which can be functionally combined. Whereas the piezoelectricity enables realisation of an integrated coupling mechanism, the 2DEG additionally delivers a pronounced sensitivity to mechanical stress and charge, which allows the sensor to use the pyroelectric effects. The dynamic change in 2DEG conductivity is also caused by a change in piezoelectric polarisation.

The source and drain contacts connecting the PC-HEMT to the electric circuit may be ohmic or non-ohmic (capacitively-coupled, as will be described below). In one embodiment, FIG. 10c shows a cross-sectional (XZ) view (A-A) of the transistor of the present application, comprising:
(a) a multilayer heterojunction structure composed of III-V single-crystalline or polycrystalline semiconductor materials, said structure comprising at least one buffer layer (11) and at least one barrier layer (12), said layers being stacked alternately, and said structure being placed on free-standing membranes (18);
(b) a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current, respectively, in said transistor between non-ohmic source and drain contacts;
(c) electrical metallisations (14) capacitively-coupled to said 2DEG channel (13) for inducing displacement currents (25), thereby creating non-ohmic source and drain contacts connecting said transistor to an electric circuit; and
(d) a toroidal metamaterial electrode (16) placed on a top layer between said non-ohmic source and drain contacts in the open gate area (17) of the transistor, said toroidal metamaterial electrode (16) is capable of detecting electrical signals in the frequency range between 30 GHz to 300 THz;
characterised in that the thickness (d) of the top layer of said heterojunction structure in said open gate area (17) is 5-9 nm which corresponds to the pseudo-conducting current range between normally-on and normally-off operation mode of the transistor, and the surface of said top layer has a roughness of about 0.2 nm or less.

“Capacitive coupling” is defined as an energy transfer within the same electric circuit or between different electric circuits by means of displacement currents induced by existing electric fields between circuit/s nodes. In general, ohmic contacts are the contacts that follow Ohm's law, meaning that the current flowing through them is directly proportional to the voltage. Non-ohmic contacts however do not follow the same linear relationship of the Ohm's law. In other words, electric current passing through non-ohmic contacts is not linearly proportional to voltage. Instead, it gives a steep curve with an increasing gradient, since the resistance in that case increases as the electric current increases, resulting in increase of the voltage across non-ohmic contacts. This is because electrons carry more energy, and when they collide with atoms in the conducting channel, they transfer more energy creating new high-energy vibrational states, thereby increasing resistance and temperature.

When electrical metallisations are placed over single-crystalline or polycrystalline semiconductor material, the “Schottky contact” or “Schottky barrier contact” between the metal and the semiconductor occurs. Energy of this contact is covered by the Schottky-Mott rule, which predicts the energy barrier between a metal and a semiconductor to be proportional to the difference of the metal-vacuum work function and the semiconductor-vacuum electron affinity. However, this is an ideal theoretical behaviour, while in reality most interfaces between a metal and a semiconductor follow this rule only to some degree. The boundary of a semiconductor crystal abrupt by a metal creates new electron states within its band gap. These new electron states induced by a metal and their occupation push the centre of the band gap to the Fermi level. This phenomenon of shifting the centre of the band gap to the Fermi level as a result of a metal-semiconductor contact is defined as “Fermi level pinning”, which differs from one semiconductor to another. If the Fermi level is energetically far from the band edge, the Schottky contact would preferably be formed. However, if the Fermi level is close to the band edge, an ohmic contact would preferably be formed. The Schottky barrier contact is a rectifying non-ohmic contact, which in reality is almost independent of the semi-conductor or metal work functions.

Thus, a non-ohmic contact allows electric current to flow only in one direction with a non-linear current-voltage curve that looks like that of a diode. On the contrary, an ohmic contact allows electric current to flow in both directions roughly equally within normal device operation range, with an almost linear current-voltage relationship that comes close to that of a resistor (hence, “ohmic”).

Thus, FIG. 10c illustrates the situation when an electrical connection of the transistor to the 2DEG channel is realised via capacitive coupling to the electrical metallisations through a Schottky barrier contact. This coupling becomes possible only if sufficiently high AC frequency (higher than 30 kHz) is applied to the metallisations. In other words, if the source and drain contacts are non-ohmic, the DC readout cannot be performed. Instead, in order to electrically contact the 2DEG channel underneath, which is about 7-20 nm bellow the metallisations (14), the AC frequency regime must be used. The capacitive coupling of the non-ohmic metal contacts with the 2DEG channel is normally induced at the AC frequency higher than 30 kHz. The AC readout or impedance measurements of the electric current flowing through the 2DEG channel are carried out on that case. The electrical metallisations capacitively coupled to the 2DEG channel utilise the known phenomenon of energy transfer by displacement currents. These displacement currents are induced by existing electrical fields between the electrical metallisations and the conducting 2DEG channel operated in the AC frequency mode through the Schottky contact as explained above.

Instead of creating the non-ohmic contacts, the source and drain areas may be highly-doped. In that case, the strong doping of the source and drain areas may result in a band-edge mismatch. However, if the semiconductor is doped strongly enough, it will form a certain potential barrier, low enough for conducting electrons to have a high probability of tunnelling through this barrier, and therefore conducting an electric current through the 2DEG channel Thus, an electrical connection to the 2DEG channel can be realised with highly doped semiconductor areas overlapping the 2DEG channel and having a very low electrical resistance. Dopant ions such as boron (B⁺), phosphorus (P⁺) or arsenic (As⁺) are generally created from a gas source, so that the purity of the source can be very high. When implanted in a semiconductor, each dopant atom creates a charge carrier in the semiconductor material after annealing. Holes are created for a p-type dopant, and electrons are created for an n-type dopant, modifying conductivity of the semiconductor in its vicinity. As⁺ can be used for n-type doping, while B⁺ and P⁺ ions can be used for p-type doping. For example, in case of the AlGaN/GaN structure, the source and drain areas of the silicon structure are heavily doped with either B⁺ or P⁺ to create an electrical connection to the 2DEG channel. The silicon layers have a very low electrical junction resistance between each other in that case, and in order to induce an electrical current in the 2DEG channel, the metallisations are placed on top of the source and drain areas and connected to a circuit.

The third option would be the use of the photo effect that may also induce an electric current in the 2DEG channel. In order to couple the light excitation with the electronic effects in the conductive 2DEG channel, a photo effect in a silicon layer should be created. Regarding the direct photo effect, it is well known that light can only be absorbed when the energy of the absorbed photon (E=hv) is large enough for an electron to be excited into the valence band. In that case, E is the photon energy, h is Planck's constant and v is the frequency of the photon. The frequency is coupled to the wavelength λ of light by the constant speed of light c=λv. Typically the bandgap of silicon at room temperature is 1.12.eV, which means that silicon becomes transparent for wavelength larger than 1240 nm, which is the near infrared range.

For smaller wavelength (i.e. larger energy of the photons), electron/hole pairs are generated leading to a photocurrent. In the fully-depleted, intrinsically doped silicon structures, this results in a higher charge carrier density and consequently, higher sensitivity. For these structures, light is adsorbed in the whole visible range making such devices ideal photodetectors. The mechanism that allows the silicon semiconductor to become photosensitive to irradiation with light has already been described in literature. In the direct photo effect, it can be tuned by the size, crystalline direction and surface termination. These effects actually originate from two-dimensional quantum confinement of electrons in the nano-sized 2DEG structure.

Although irradiation of the silicon structure with light of larger wavelengths with photon energies below the bandgap does not have enough energy to excite carriers from the valence to the conduction band in bulk silicon, the electron/hole pairs can also be generated between the valence band and surface states, and the donor-like surface trap states can still be formed (see the definition and explanation of the surface trap states below). The electrons actually deplete these holes trapped at the surface and hence, modulate the gate field. The photogenerated holes are confined to the centre of the silicon structure by the gate field, where they increase the conduction of the 2DEG channel, because of the band bending. The holes increase the channel conductivity for a certain lifetime until they are trapped (recaptured) at the surface. The gain of the transistor can be extremely huge if this re-trapping lifetime is much longer than the holes transit time.

The transistors of the present invention shown in FIGS. 3, 10a and 10c may further comprise a dielectric layer, which is deposited on top of the heterojunction structure. The optional dielectric layer is used for device passivation and can be made for example, from the SiO—SiN—SiO (“ONO”) stack having thickness of 100-100-100 nm, or alternatively, from the SiN—SiO—SiN (“NON”) stack having the same thicknesses. This dielectric layer is deposited on top of the heterojunction structure by a method of plasma-enhanced chemical vapour deposition (PECVD), which is a stress-free deposition technique.

In a particular aspect of the present invention, a metamaterial electrode (16) is placed on a top layer between the source and drain contacts in the open gate area (17) of the transistor. This metamaterial electrode (16) is capable of detecting electrical signals in the sub-THz or THz frequency range, as will be discussed below. In a specific embodiment, the metamaterial electrode (16) has a toroidal shape. The plurality of the toroidal metamaterial electrodes (16) used in the present invention is defined as an “Aharonov-Bohm antenna”, which can detect signals in the sub-THz and THz frequency range. By applying additional plasmonic filters of any kind, the frequencies could be tuned precisely to a specific frequency of choice. In general, the instant Aharonov-Bohm antenna is a co-planar broadband-antenna, which is made from a metamaterial in a form of multiple toroids. It is a reciprocal device and collects in a passive mode exactly the same frequencies that can be actively radiated. Due to its very broadband character, the Aharonov-Bohm antenna may receive signals in a broad range between 30 GHz to 300 THz.

The Aharonov-Bohm antenna may be realised on a thin dielectric substrate over the top layer of the transistor. Advantages of the Aharonov-Bohm antenna is its broadband characteristics suitable for ultra-wideband signals in the sub-THz and THz frequency domain, its relatively easy manufacturing process using common methods for the PCB production, and its easy impedance matching to the feeding line using microstrip line modelling methods. Also, the Aharonov-Bohm antenna has been chosen because it permits to integrate a long meander delay without having undesired effects.

In general, “metamaterial” is an arrangement of artificial structural elements, which are defined herein as “metamolecules”, designed to achieve advantageous and unusual properties unattainable in natural media. A metamaterial gains its properties from its unique structure rather than composition. Examples of metamaterials are aerogel, carbon nanotubes, mother-of-pearl gets its rainbow colour from metamaterials of biological origin, bubble wrap is a favourite packing-based stress reliever that can absorb masses of energy except made from aluminium, titanium foam clinging to the shape of a simple foam and perfect for replacing human bones, molecular superglue, amorphous metals with a disordered atomic structure made by quickly cooling molten metals, fibroin-based artificial spider silk, D3o polyurethane-based energy-absorbing material, bioluminescent bacteria-based glowing materials, graphene and graphene aerogel.

A separate class of metamaterials is the one with the toroidal response. The toroidal observation is mediated by the excitation of currents flowing in inclusions of toroidal metamolecules and resembles poloidal currents along the meridians of gedanken torus. The destructive interference between the toroidal and electric dipole moments leads to lack of the far-fields but the fields in the metamolecules origin describing by δ-function. Such field configuration, referred as a toroidal dipole or anapole, corresponds to the field of a solenoid bent into a torus and allows observing the phenomenon of electro-magnetically-induced transparency, provides an unusually high Q-factor in metamaterials, enables a cloaking for nanoparticles, and confirms the dynamic Aharonov-Bohm effect. Basharin et al in “Extremely high Q-factor metamaterials due to anapole excitation”, Phys. Rev. B 95, 2017, 035104, demonstrated that the anapole excitation in planar metamaterials, which enabled an extremely high Q-factor in microwave, gives promising opportunities for tuneable metamaterials due to the strong electromagnetic fields' localisation within metamolecules.

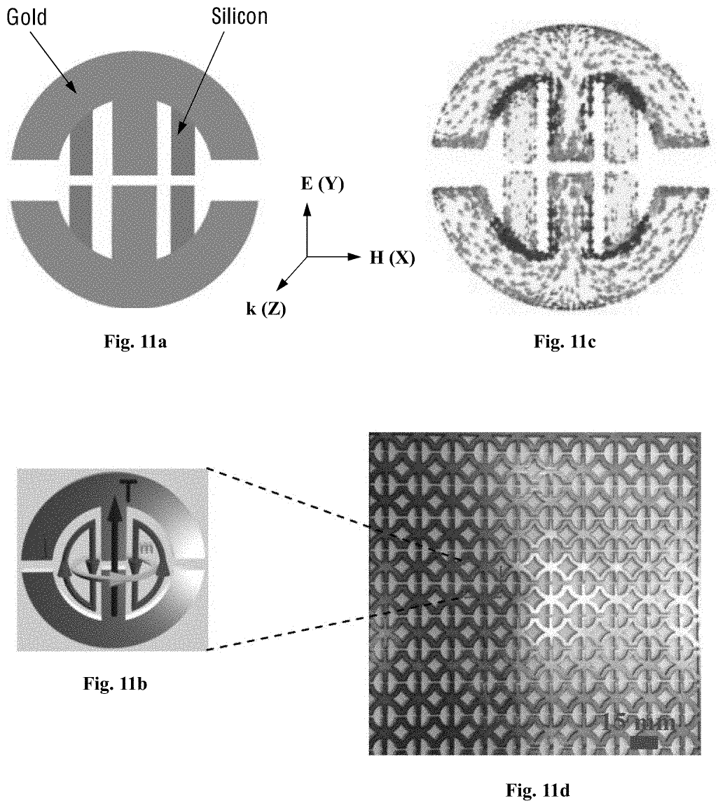

The Aharonov-Bohm antenna of the present invention is based on the design of the toroidal metamaterial suggested by Basharin et al (2017) in a tuneable regime. The toroidal metamaterial of the present invention consists of toroidal metamolecules made of photoconductive silicon capable of transiting from dielectric to metallic state and having the response in the sub-THz and THz frequency domain. As shown in FIGS. 11a-11c, the meta-molecules constituting the toroidal metamaterial, shown in FIG. 11d, contain two split parts. The incident plane electromagnetic wave with electric field E aligned with the central wire excites conductive currents in each loop of the metamolecule. The currents schematically shown in FIGS. 11b-11c form a closed vortex of magnetic field H. Each current induces the circulating magnetic moments m wreathing around the central part of the metamolecule. Such configuration of electro-magnetic fields supports the toroidal dipole excitation with a toroidal moment T as a result, oscillating upward and downward within the metamolecule along the Z axis. However, two side gaps also support a magnetic quadrupole moment Q. Moreover, due to the central gap electric moment P can be excited in the metamolecule, the electric dipole also arises in the metamolecule and maintains the anapole mode, and the central gap becomes a necessary part of anapole in accordance with the destructive interference between electric and toroidal dipole moments. The advantage is a very narrow line in the transmission spectrum of a metamaterial. Based on this, the planar toroidal metamaterial is used as a building block for sub-THz and THz modulators in the present invention.

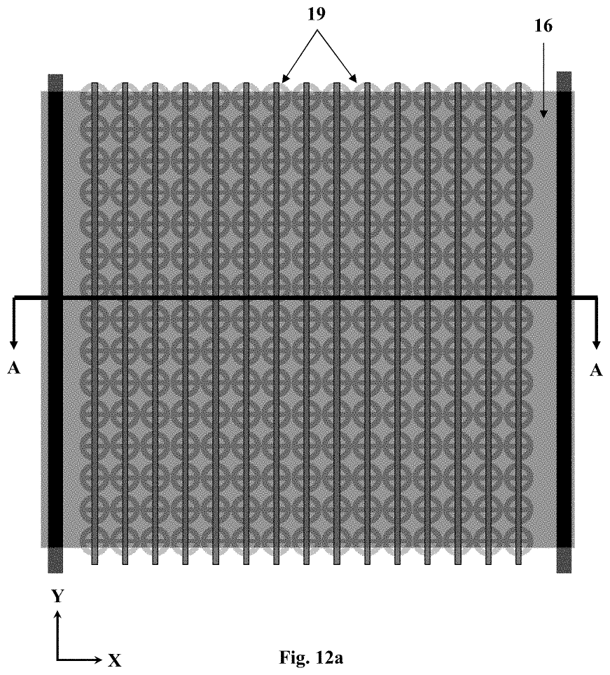

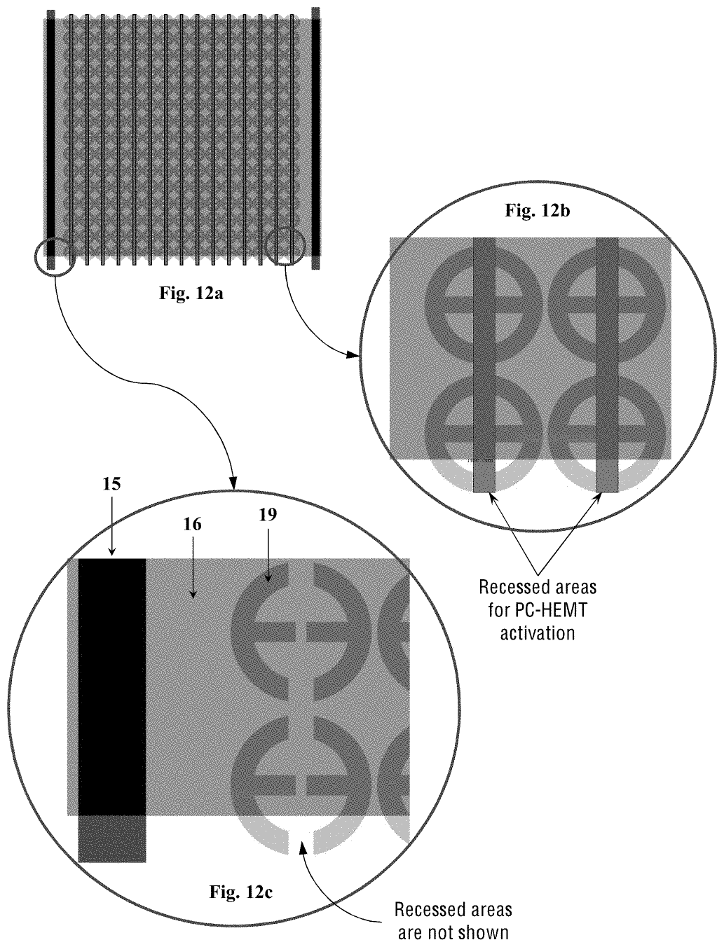

Reference is now made to FIG. 12a schematically showing the top (XY) view and the basic topology of the transistor of the present invention. The heterojunction structure is coated with the metamaterial (16) composed of the toroidal metamolecules (19) arrayed in a periodic structure. FIGS. 12a-12c demonstrate the first configuration of the PC-HEMT of the present invention. As explained above, such toroidal metamaterial (16) realising the Aharonov-Bohm phenomenon has very unique properties for interaction with electromagnetic radiation. This structure shown in FIGS. 12a-12c creates a wave separation between magnetic and electric fields, and thus acts partly as a metamaterial surface with a negative refractive index. Another key property of this configuration is its ability to strongly concentrate the electric field at specific “sweet spots” of the structure making it possible to use this structure as a gating structure on top of the 2DEG channel of the transistor. The interaction of the toroidal metamolecules array of this configuration with the sub-THz or THz radiation at specific frequency is dependent on the lateral sizes of each metamolecule (19) and on the distance between them in the array.

The combination of lateral sizes and distances between the metamolecules (19) in the array enable negative refractive-index for specific sub-THz and THz wavelengths. If the interaction between the sub-THz or THz-radiation and the gate created by the array of the toroidal metamolecules (19) is at its strongest point, the electric field density at the sweet points of any particular metamolecule in the array is strongly changing, thereby providing a gating effect in both DC and RF modes. If many transistors with the toroidal metamaterial (16) are used in the signal detection, the sub-THz or THz spectrum can be detected and recorded. Moreover, if the PC-HEMTs of the present invention are coated with chemical or bimolecular specific layer, sensors based on such PC-HEMTs can be used as selective biochemical sensors, which is another aspect of the present invention that will be described below.

Thus, the configuration shown in FIGS. 12a-12c allows to effectively couple the toroidal metamaterial on top of the heterojunction structure with the 2DEG channel underneath.

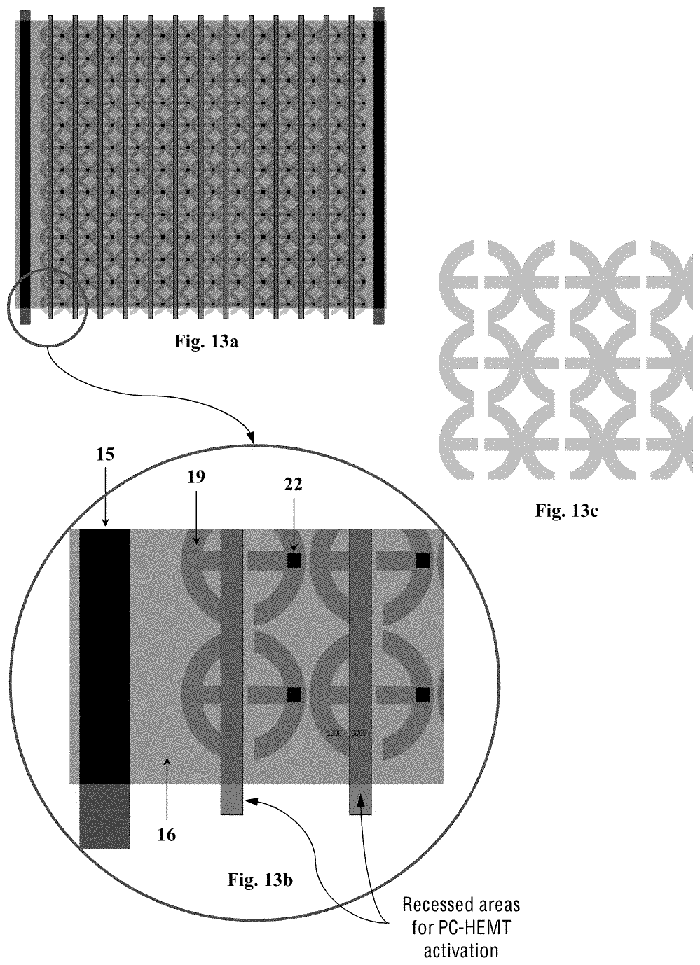

Reference is now made to FIGS. 13a-13c showing the second configuration of the PC-HEMT of the present invention. In this configuration, there is a connection to each toroidal metamolecule (19) in the array via its dedicated ohmic point contact (22). The metamolecules (19) are generally Schottky-metal gate structures creating a strong electric and magnetic field at the sweet spots. By connecting this array of the metamolecules (19) with the conducting 2DEG channel electrically, it is possible to create much stronger potential difference at the interface between the 2DEG channel and each metamolecule (19) in the array. This is because the metamolecules (19) when irradiated with sub-THz or THz radiation, build up a potential difference between their two parts, as explained above. With the electrical connection the potential difference of electric field can be coupled much stronger.

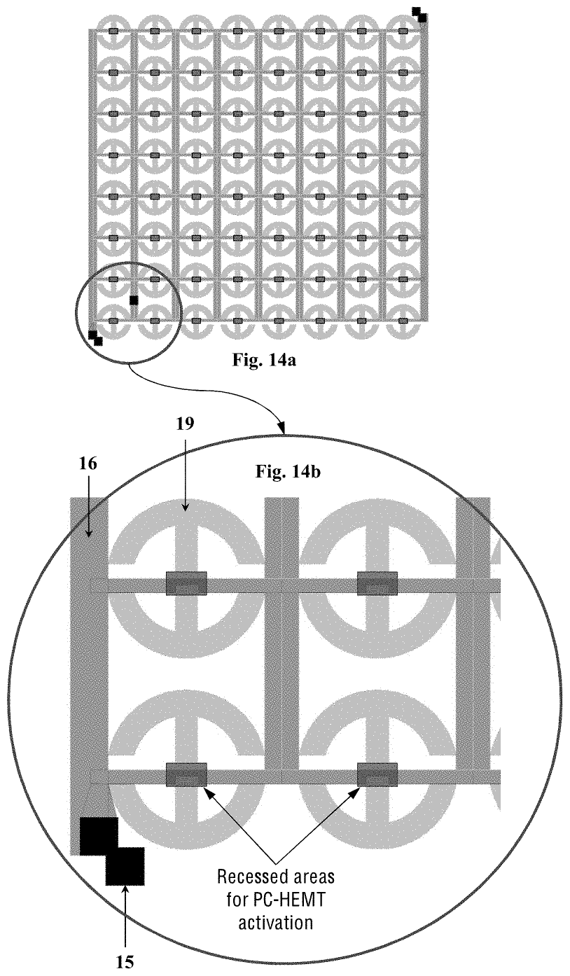

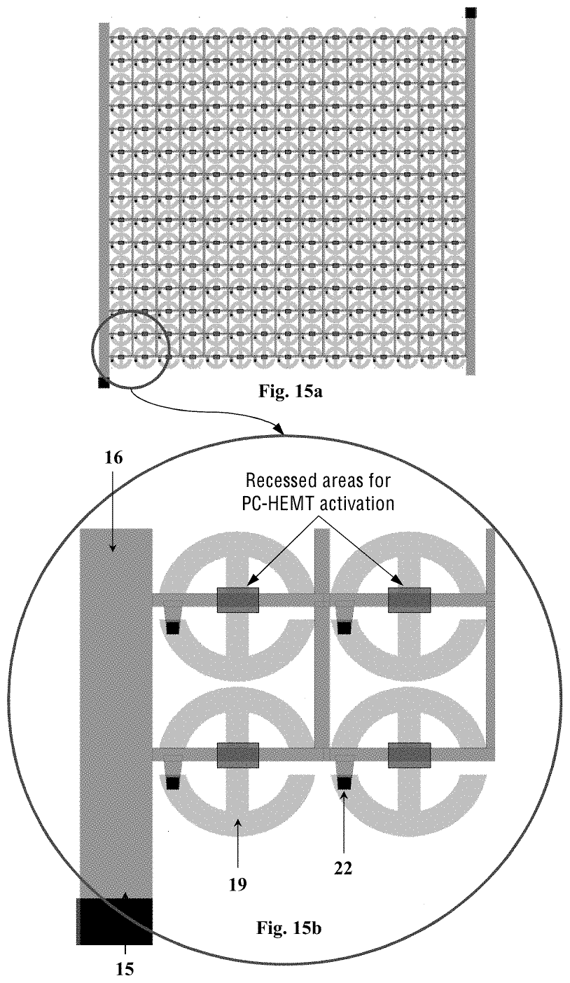

FIGS. 14a-14b show the third possible configuration of the PC-HEMT of the present invention. This configuration represents a different way of the channel gating between the conducting 2DEG channel and the PC-HEMT using the toroidal metamolecules array. In this configuration, the 2DEG channel is patterned in order to strongly increase the interaction between the Schottky-Aharonov floating metamolecules in the array at the recessed PC-HEMT areas.

The fourth configuration of the PC-HEMT shown in FIGS. 15-15b has the same layout as the third configuration shown in FIGS. 14a-14b, but with an additional dedicated ohmic point contact (22) to each metamolecule in the array as in the second configuration shown in FIGS. 13a-13c.

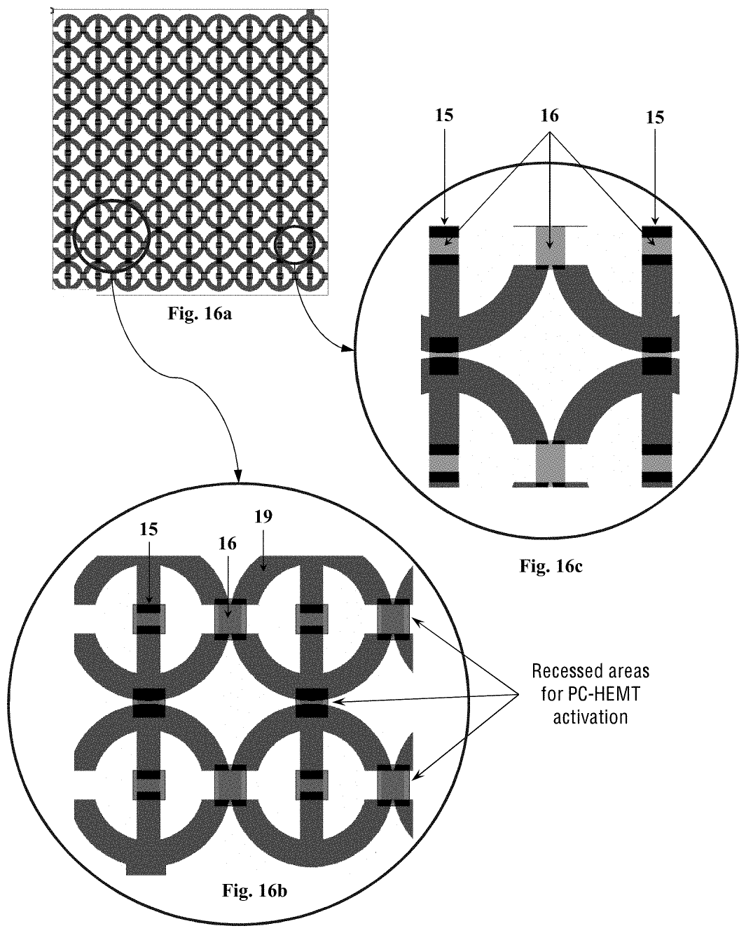

The last (fifth) possible configuration shown in FIGS. 16a-16c uses only the patterned 2DEG channel created between the ohmic contacts. The top layer of the hetero junction structure is recessed in the areas where the metamolecules contact each other.

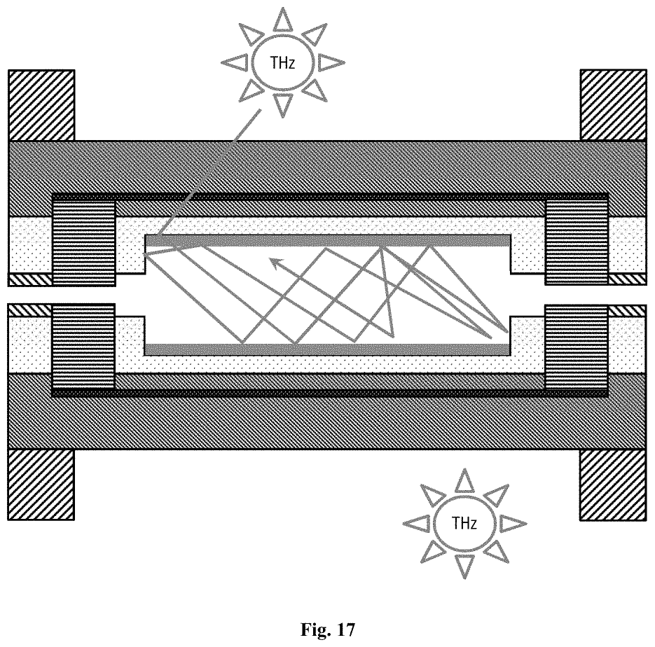

Reference is now made to FIG. 17 showing a microelectronic sensor of the present invention for sensing electrical signals in the sub-THz and THz frequency range. In a particular aspect, the microelectronic sensor comprises at least one pair of the PC-HEMTs of the present invention, characterised in that said transistors are installed with their open gates facing each other and thereby forming a resonant cavity enclosed by their open gates, where said resonant cavity is capable of maintaining resonance in a sub-THz or THz frequency range.

In another embodiment, the transistor of the present invention further comprises at least one (bio)molecular layer immobilised on the surface of the metamaterial electrode (16) within the open-gate area (17) for sensing target chemical compounds or biomolecules. The (bio)molecular layer allows sensing for example, gas molecules to be bound or adsorbed and then detected. This (bio)molecular layer may further increase sensitivity and selectivity of the sensor based on the transistor of the present invention. The (bio)molecular layer is composed, for example, of polymers, redox-active molecules, such as phthalocyanines, metalorganic frameworks, such as metal porphyrins, for example hemin, biomolecules, for example receptors, antibodies, DNA, aptamers or proteins, water molecules, for example forming a water vapour layer, such as a boundary surface water layer, oxides, semi-conductive layer or catalytic metallic layer. The (bio)molecular layer is immobilised over either a portion of the metamaterial electrode (16) surface or substantially over its entire surface in the open-gate area (17) to further improve sensitivity of the sensor for detection of target molecules or analytes.

In general, the (bio)molecular specific layer is any suitable coating that adsorbs selected chemicals or biomolecules present in the environment. The presence of molecules or biomolecules adsorbed on this (bio)molecular layer modulates the signal received by the transistor due to shifting equilibrium between the 2DEG/2DHG channel density and the number of ionised surface donors which is governed by charge neutrality and continuity of the electric field at interfaces. Measurement of this modulation is used to indicate the identity and concentration of specific molecules or biomolecules in the environment.

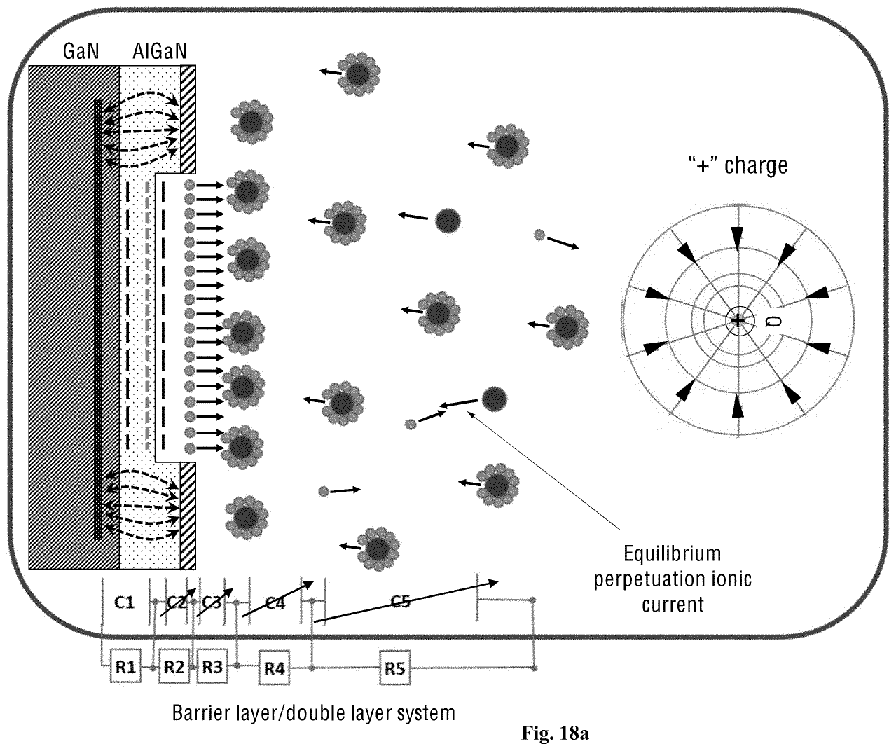

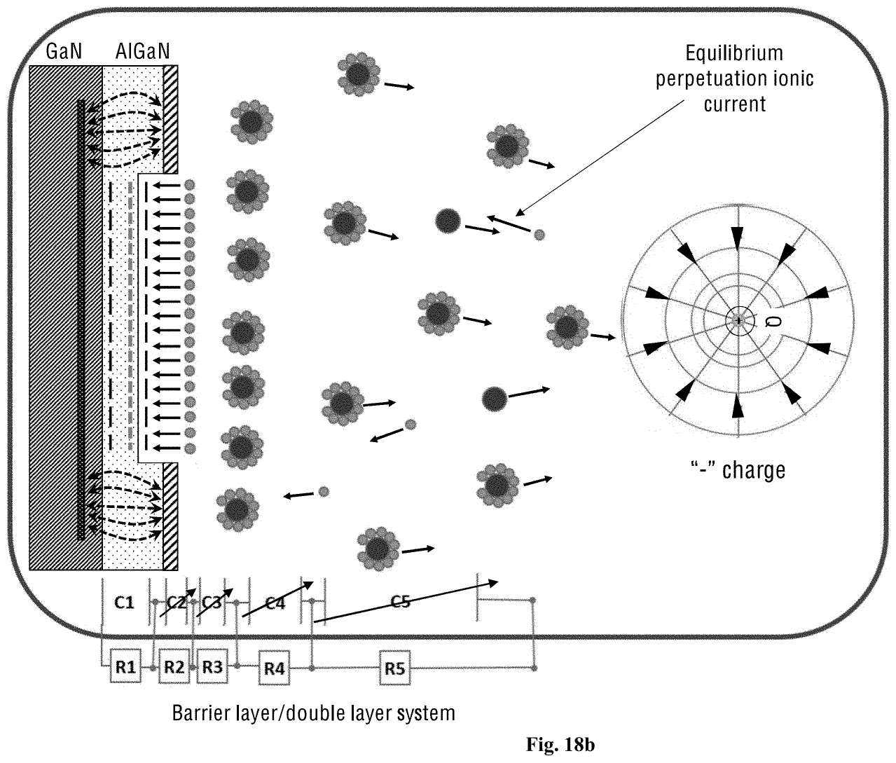

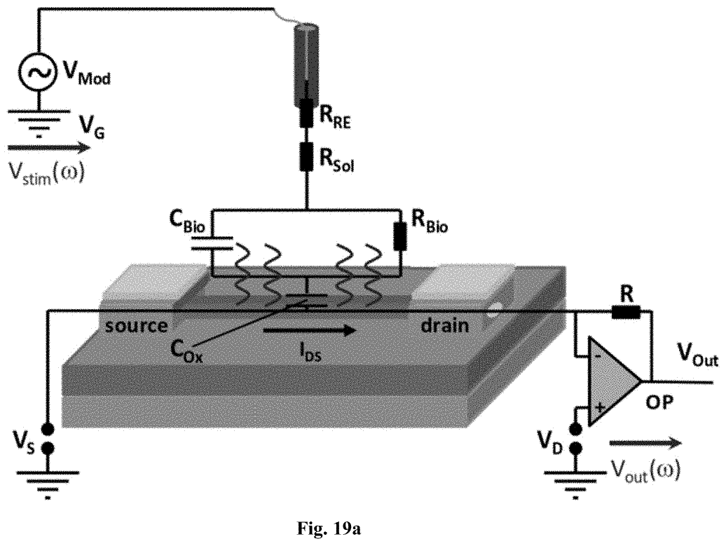

Reference is now made to FIGS. 18a-18b illustrating the barrier layer/liquid or gas interface with the double layer formation, simplified equivalent interface circuitry and ion electrodynamics during exposure of the sensor to a positive charge (FIG. 18a) and a negative charge (FIG. 18b). When immersed into a gas or liquid environment, any surface potential causes natural formation of an electrochemical double layer at the contact interface to maintain charge equilibrium between the solid state and ionic conductive liquid or gas.

In FIGS. 18a-18b, this double layer is shown together with the simplified equivalent circuitry at the interface. The double layer is created with a 1- to 3-nm-thick sharp separation between the negative and positive ion space charge zones C2-R2 and C3-R3, which cause a secondary space charge equilibrium zone C4-R4 (10 nm to 1 μm) and charge gradient zone C5-R5 disappearing in the bulk liquid or gas. When there is no more potential shift from the solid and from the liquid or gas, then the charge equilibrium is maintained with C1/R1-C5/R5 elements possessing quasi-constant values.

Ion flow is schematically shown in FIGS. 18a and 18b with vector arrows during an electrodynamic rearrangement when an external charge is introduced into an equilibrated electrolyte. FIG. 18a shows the electrodynamic rearrangement with an external positive charge, and FIG. 18b shows the electrodynamic rearrangement but with an external negative charge. When the ions react to an external electric field applied in the liquid, the equivalent circuitry mirroring the space charges changes accordingly. Since the PC-HEMT of the present application is extremely sensitive to any smallest surface charge changes (C1/R1) due to its pseudo-conductivity, as explained above, rearrangement of the gradient ions in the shown space charge zones from C5/R5 to C2/R2 is capable of modulating the 2DEG conductivity. Dynamics and magnitude of the newly formed equilibrium at each time moment is directly proportional to the liquid electrolyte conductivity, ions mobility and external charge value, therefore defining the resulting electrolyte charge. In general, any electrolyte strongly enhances the sensor charge response due to the excellent direct charge transfer towards the barrier layer/electrolyte interface. The ions of the liquid or gas interact directly with the super sensitive surface trap states of the ultrathin barrier layer.

The above phenomenon occurring at the PC-HEMT surface, discovered by the present inventors, is defined as an “intra-fluid ionic interaction”. Thus, if this transistor connected to a circuit is immersed into an ion conductive fluid (being liquid or gas), then ions of the fluid start electro-dynamically react to any external charge by their movement. Being in direct contact to the barrier layer surface, the charge sensitivity is tremendously enhanced. The fluid acts in this case as an additional antenna (additional to the metamaterial toroidal electrode antenna) perfectly matching the 2DEG transducer. Electric charges generated in any environment, as well as their super position dipole, are projected to this fluid antenna, in which the transistor is immersed.

To sum-up, in direct current (DC) mode, the top layer-to-fluid-interface of the transistor is in charge equilibrium, where the 2DEG is directly incorporated as a balancing polarisation element. Once an external electric charge (originated from dipole molecules forming a layer or two neutral molecules creating a dipole pair under London forces) is introduced into the electrolyte fluid environment, the net charge equilibrium is shifted resulting in a change in the electron density and mobility. In case of the 2DEG channel, it becomes easily modulated and the strongest amplification phenomenon is observed. Sensitivity of the PC-HEMT in this case is so ultra-high that it allows detecting neutral molecules diffusing to the surface and coupling to the top layer surface of the transistor via a getter effect changing the surface trap states. The getter effect actually allows the sensor based on the PC-HEMT of the present invention to collect free gases by adsorption, absorption or occlusion.

In radiofrequency (RF) mode, when the electric current in the conducting 2DEG channel is alternating (AC), the near-field and displacement current coupling effects at electrochemical double layers take place. In that case, the super-Debye interactions allow detection of any ion types selectively at MHz frequency range and ion solvation shells and resonance frequencies of intra-fluid ion-ion interaction at GHz range.

As discussed above, at any solid state/electrolyte interface, the capacitive and resistive elements of the sensor form an electrochemical surface potential originated from an interaction between the surface trap states and a double layer capacity, while the interaction between the pseudo-conducting 2DEG and the surface trap states originates from tunnelling and electrostatics. It has now been surprisingly found that operation of the PC-HEMT sensor as an open gate field-effect transistor is not required in order to modulate the surface electrochemical potential within the barrier layer/electrolyte system.

It has recently been found by the present inventors that the PC-HEMT of the invention is capable of overcoming the Debye length limitation. The overall design of the transistor enables its additional operation in the frequency domain and helps to stabilise the electronic readout when recording very small DC current changes. Therefore, the sensors of the present invention based on the PC-HEMT can be used in impedance spectroscopy applications. Combination of potentiometric and impedimetric readout enables a more reliable sensing of molecules with the potential to sense beyond the Debye screening of electrical charges in an electrolyte solution, which is usually the limiting factor in most of the sensors having only potentiometric or conductometric readout.

As mentioned above, the PC-HEMT of the present invention is functionalised optionally with different molecules (receptors), which are capable of binding to a target (analyte) molecule, for sensing. As a result, the PC-HEMT-based sensor of the invention can be used for label-free detection of target (analyte) molecules by monitoring changes in the electric current of the transistor caused by variations in the charge density or the impedance at the open gate-electrolyte interface.

In general, charges formed in a liquid medium sensed by ISFETs come from the dissolved molecules. Depending on the pH value of the liquid and the molecules' isoelectric point, the dissolved molecules exhibit a global charge. However, this charge may be non-uniformly distributed over the molecule. In addition, the molecules have different sizes and different structures. Therefore, it is very important that:
the sensor's interface is chemically engineered in a very uniform and reproducible manner,
receptors need to be immobilised on the sensor's surface as highly selective receptor layer with a very uniform grafting density,
the sensor should have redundant structure exhibiting multiple sensors cancelling out wrongly functionalised transistors, and
a molecular friendly surface architecture and microenvironment with fixed pH value, fixed ionic strength and temperature needs to be established to avoid denaturation of the molecules on the sensor surface.
   The latter is controlled by respective reference sensors for temperature, pH value and ionic strength in the sensor chip design. However, even with the above-mentioned ideal sensor design it can be the case that the potentiometric detection of charges, which lead to changes in surface potential and hence, to a shift of the ISFET threshold voltage, cannot be detected, because the relevant charges are located outside the Debye screening length of the liquid electrolyte.

In most cases (in most biosensors), molecular receptors bound to the transistor surface are spatially separated from this surface by molecular cross-linkers or proteins of about 5-15 nm length. Therefore, the aforementioned charges are screened from the sensing surface by dissolved counter ions. As a result of the screening, the electro-static potential that arises from charges on the analyte molecule exponentially decreases to zero with increasing the distance from the sensing surface. This screening distance is defined as a “Debye length”, and it must be carefully selected when designing the receptor layer of any ISFET in order to ensure the optimal sensing. For example, when detecting molecules in blood serum, the typical Debye screening length is 0.78 nm at temperature 36° C. with an electrical permittivity of 74.5 for water. This means that after this length, the Debye screening length is given by:

$${λ}_{D} = \sqrt{\frac{{ϵ}_{r} {ϵ}_{0} {k}_{b} T}{2 {n}_{0} {z}^{2} {e}^{2}}}$$

where n₀ is the bulk concentration of the electrolyte, ϵ₀ is the relative dielectric permittivity of the solvent (in case of water at 36° C. a value of 74.5), co is the permittivity of the vacuum, k_{b} is the Boltzmann constant, T is the temperature, z is the ion charge, and e is the elementary charge.

The screening length means that an electrical field originating from a point charge is dropped to its 1/e value (29%) in this length. Because of this limitation, charges from larger biomolecules (5-15 nm) cannot be detected in a serum sample. To overcome this problem, the charges should be attracted closer to the sensor surface by using very short length receptors or by operating the sensor in completely desalted buffers for electronic molecular detection.

Thus, the Debye length limitation can be overcome by modification of the receptors and controlling the immobilisation density over the ISFET's sensing surface. Roey Elnathan et al 2012 (in “Biorecognition Layer Engineering: Overcoming Screening Limitations of Nanowire-Based FET Devices”, Nanoletters 12, 2012, pp. 5245-5254) described this approach in detail and demonstrated the increased sensitivity of their sensor to troponin detection directly from serum for the diagnosis of acute myocardial infarction. However, the present inventors made a step forward and proposed to sense beyond the Debye screening length without any modification of the receptors, but operating the PC-HEMT of the present invention at high frequencies and using a combined transducer principle, which is one of the aspects of the present invention. It will be described below in details. By combination of a precise monitoring and control of the main parameters, temperature, pH and ionic strength with an array of electronically identical PC-HEMTs of the present invention, and a highly reproducible and uniform bio-receptor surface layer, the precise identification of biomolecules can be obtained.

By using coatings with selective adsorption properties, sensors detecting specific chemical or biological compounds for both gas-phase and liquid-phase environments have been now developed by the present applicants. Typically, durable oxide-based coatings that are chemically modified to provide the required adsorption characteristics are used. These coatings can selectively adsorb ionic species from solution for use in applications such as monitoring electroplating processes or waste streams for toxic metals such as chromium, cadmium, or lead.

Polymer coatings that adsorb a wide variety of chemicals are ideally suited for monitoring the highly regulated ozone-depleting chlorinated hydrocarbons. Simultaneous measurement of the wave velocity and attenuation can be used to identify chemical compounds and their concentration. One of the applications of the sensors of the present invention is the selective detection of organophosphates, which are a common class of chemical warfare agent. The detection of these chemicals is done by the active chemical layer composed of thin films of self-assembled monolayers. The sensitivity of these films on the piezoelectric material of the sensor endows the sensor with immunity to interference from water vapour and common organic solvents while providing sensitivity in the part-per-billion concentration of organophosphates. As a result, arrays of such sensors with appropriate coatings can be used to detect the production of chemical weapons.

Another application of the sensors of the present invention is a chemical detection and analysis of environmentally toxic compounds and toxins, such as food toxins, for example aflatoxin, neurotoxic compounds, for example lead, methanol, manganese glutamate, nitrix oxide, Botox, tetanus toxin or tetrodotoxin, shellfish poisoning toxins, for example saxitoxin or microcystin, Bisphenol A, oxybenzone and butylated hydroxyanisole. In general, chemical detection and analysis of toxic compounds can be aimed at determining the level or activity of these compounds in the emission sample (into which the toxic compound is incorporated en route to human exposure, for example in industrial effluents), in the transport medium (for example, air, waste water, soil, skin, blood or urine), and at the point of human exposure, for example in potable water. Sensing the emission sample, the transport medium, and the point of human exposure may be necessary for a comprehensive plan designed both to detect toxic compounds, analyse them and to exert control on the emission of the toxic compounds in order to achieve hazard reduction.

For a given toxic analyte, chemical sensors of the present application will certainly differ in sensitivity, selectivity, or other characteristics, which may be required to monitor the emission sample, the transport medium, and individual exposure. Concentration of a toxic compound is typically greater in the emission sample than after dispersal in a transport medium and can vary widely. The physical and chemical properties of the analyte and its immediate environment (airborne vapour, contained in solid or liquid aerosol, chemically or photochemically reactive and decomposing into compounds of different toxicity, radioactive, ionic, acidic or lipophilic) are also influential in the design of a suitable configuration for the sensor of an embodiment.

Still another application of the sensors of the present invention is a chemical detection of explosives. In general, a large range of explosives can be detected with the sensor of an embodiment. A distinction is made between the bulk explosives and the trace explosives. In case of the trace explosives, the sensor is capable of detecting vapours of the explosive chemicals, thereby detecting the trace quantities emitted from explosive materials either directly in the environment or in the particulates of explosive materials that have been collected and then vaporised in the laboratory within the analytical instrument. The sensor of an embodiment can be operated both by direct sampling of the air containing the trace explosive vapours as well as by vaporising a sample that was collected by swiping a surface contaminated with explosive particulates.

Apart from simply being able to detect explosive materials, the sensor of the present invention is capable of identifying and quantifying the explosives. In general, a sensor that is used as a safety measure at airports will have other requirements than one that will be used in the field during military missions. Therefore, the configuration of the sensor can vary dependent on the particular application. There are different requirements to the throughput and, because of elevated background levels in military environments, the dynamic range. Furthermore, the military sensor for detection and analysis of explosives should be portable compared to the fixed sensors in laboratories or airports. Another consideration is the difference between detection and identification. In some instances, a device will be used to sense whether a certain explosive material is present, whereas in others it is also necessary to determine which explosive compound it is. Furthermore, it can be important to consider how many different compounds, or groups of compounds, one device must be able to detect or identify. Different sensor configurations described below meet the above requirements for different types of the sensors.

Instead of detecting the explosive compounds themselves, the sensors of the present invention may also be used to detect other materials that could indicate the presence of an explosive material. These “other” materials are actually associated compounds that tend to be present when explosives are present, such as decomposition gases or even taggants, materials that have been added during the production of the explosive to facilitate the detection. An advantage of this approach is that taggants and some associated compounds have a higher vapour pressure than the explosive compound itself and are thus easier to detect. In addition to the sensitivity, the selectivity of the sensor should also be considered. The selectivity of the sensors of an embodiment to vapours of the trace explosives may be increased by using them in an array. By using the sensors in an array, it is possible to obtain a signal similar to an artificial olfactory system of a nose when the responses of a number of sensors are combined to give a fingerprint-like signal. In this case, pattern recognition methods, such as multiple axes radar plots, can be used to analyse the signal, match it to known responses from a database, and thus identify the explosive.

Examples of the explosive materials detected by the sensors of the invention in aqueous medium are picrates, nitrates, trinitro derivatives, such as 2,4,6-trinitrotoluene (TNT), 1,3,5-trinitro-1,3,5-triazinane (RDX), N-methyl-N-(2,4,6-trinitrophenyl)nitramide (nitramine or tetryl), pentaerythritol tetranitrate (PETN), trinitroglycerine, nitric esters, derivates of chloric and perchloric acids, azides, and various other compounds that can produce an explosion, such as fulminates, acetylides, and nitrogen rich compounds such as tetrazene, octahydro-1,3,5,7-tetranitro-1,3,5,7-tetrazocine (HMX), peroxides (such as triacetone trioxide), C4 plastic explosives and ozonides. In addition to the explosives, nitrobenzene, 2, 4-dinitrotoluene and several other organic compounds were tested being concomitant chemicals of TNT or some common water pollutants. The biomolecular layer (20) can be for example, a layer of the antibodies immobilised against a specific explosive compound. Alternatively, the molecular layer can be phthalocyanine system having 2,2,3,3-tetrafluoropropyloxy substituents or cyclodextrin as sensitive materials for the detection of different explosives in aqueous media, in particular nitro-containing organic compounds.

As mentioned above, a biomolecular layer sensitive to a certain target biomolecule, such as a specific pathogen, may be deposited on the top layer surface of the PC-HEMT within the open-gate area (17). As a result, upon binding of the specific pathogen, not only an electric field change, but also a mass change is normally observed, and the density of the target molecules can be detected and further correlated to its concentration. As mentioned above, one of the embodiments of the present invention is the PC-HEMT placed on free standing membranes (18) and used in “pressure-sensitive” mode, thus being capable of measuring very small pressures (see FIGS. 10a-10b). Such sensor uses the free-standing membranes for creating a mass-loading effect which makes it possible to increase selectivity of the sensors via adding mechanical stress (mass-loading effect) as an additional parameter of the PC-HEMT-based sensor. In other words, the sensor of the present invention may also act as a miniature analytical balance, weighing the biological pathogens that bind to its surface. For example, biological pathogens may be captured very selectively by the biomolecular layer consisting of specific biological receptor molecules, such as antibodies, short peptide chains or single-strand DNAs, which are capable of distinguishing between closely related pathogens. In fact, one can think of the sensor of an embodiment as a spring with a small weight bouncing at one end. As the molecule becomes attached to the sensor, the weight on the spring increases, which causes the speed of the spring's oscillation to significantly decrease. By measuring the oscillation speed or, equivalently, the oscillation phase shift, one can determine how much of the molecule has been captured.

Thus, using the configuration of the sensor with the free-standing membranes makes is possible to increase selectivity of the sensor via adding mechanical stress (mass loading effect) of the molecular or biomolecular layer as an additional parameter of the sensor. In this configuration, the molecular or (bio)molecular layer is immobilised on the top layer of the PC-HEMT within the open-gate area (17) for the mass loading effect. Using this configuration makes is possible to increase selectivity of the sensor via adding mechanical stress (mass loading effect) of the molecular or biomolecular layer as an additional parameter of the sensor. The very flexible free-standing substrate columns-like membranes can be made in all configurations of the sensor.

Yet further application of the sensors of an embodiment is a biomolecular diagnostic including detection of DNA and proteins. In that case, the biomolecular specific layer allows proteins and DNA molecules to be bound or adsorbed and then detected. This biomolecular layer further increases the sensitivity and selectivity of the sensor of an embodiment. The biomolecular layer can be made of various capturing molecules, such as primary, secondary antibodies or fragments thereof against certain proteins to be detected, or their corresponding antigens, enzymes or their substrates, specific DNA sequences complimentary to the DNA to be detected, aptamers, receptor proteins or molecularly imprinted polymers. The biomolecular layer can be immobilised either over the surface of a portion of the recessed 2DEG/2DHG structure or over the entire surface of the PC-HEMT-like area to further improve sensitivity of the sensor for detection of a specific proteins or DNA molecules.

Alternatively, the PC-HEMT of the present invention may not be coated with any molecular layer, but still be capable of sensing target molecules or biomolecules. Since the sensor of the present invention is clearly capable of overcoming the Debye length limitation, as explained above, sensing of the electric charges with the transistor of the present invention is possible in a contactless manner, when the molecules are at some distance from the surface of the transistor. This allows to overcome a well-known “sensing noise” of any traditional biosensor having reporter molecules attached to the surface of the sensor.

As noted above, the main limitation in the DC readout mode is the Debye screening of charges. Moreover, the DC readout is not suitable for various functionalised surfaces and mainly depends on the charge carried by the target molecule. The present inventors proposed to overcome these limitations by adding the AC readout with a frequency sweep up to 1 MHz or higher. Opposite to the DC readout, the charges of the target molecules have a negligible influence on the sensing in the AC mode. Also, the AC readout can detect the presence of bound molecules. The AC sensing mode has the same basis as the impedance spectroscopy. It shows the change of the sensor's surface capacitance and resistance which contains information about the binding of the target molecule, as well as the ‘number’ (concentration) of the bound molecules.

Thus, the AC electronic readout combined with the DC readout is useful for enzymatic, electrochemical and affinity sensing when both charged and uncharged molecules are involved. When operated at higher frequencies (more than 1 MHz), the problematic Debye screening can be overcome, and also larger molecules can be sensed.

In a further aspect of the present invention, the combined transducer principle defined herein as a “triple readout” includes: DC electronic readout of the sensor, AC electronic readout of the sensor and temperature sensing. The PC-HEMT-based sensor of the present invention therefore further comprises a reference electrode and characterised with respect to its electronic properties and to the measurement configuration for molecular sensing applications. The main features of the sensors of the present invention are determined by the transfer characteristics and the output characteristics at room temperature. The transfer characteristics shows the drain current of the PC-HEMTs as a function of their source voltage at constant drain-source voltages.

In general, the term “transfer function” (TF) is a mathematical representation to describe inputs and outputs of black box models. In order to describe the frequency response of the sensor, a counter electrode and the first amplifier stage are considered as a black box element with a certain frequency response. Since the analogue transistor amplification is exploited in the present invention, the instant model is described with a term “transistor transfer function” (TTF). The TTF is defined as a mathematical ratio between the input (Vₛₜᵢₘ) and the output signal (Vₒᵤₜ) of an electrical, frequency-dependent system. Its frequency response H(jω) is defined as follows:

$$H ( j ω ) = \frac{{V}_{out} ( j ω )}{{V}_{stim} ( j ω )} ,$$

wherein ω is the angular frequency and j is the imaginary unit.

The TTF can be used to investigate impedance (defined as the ratio between voltage and applied current) or capacitance (defined as the capability of a capacitor to store charges) changes, caused by binding of molecules onto the PC-HEMT surface. This detection of analytes was reported in several publications even though the theory, on which the TTF relies, is still under discussion, because for each particular device and amplifier design, there are many parasitic side parameters that have an extremely drastic effect on the TTF. A universal model is therefore difficult to establish. However, the present inventors have demonstrated that it is possible, to investigate, for instance the DNA hybridization, protein binding and to perform cell recordings by measuring this function.

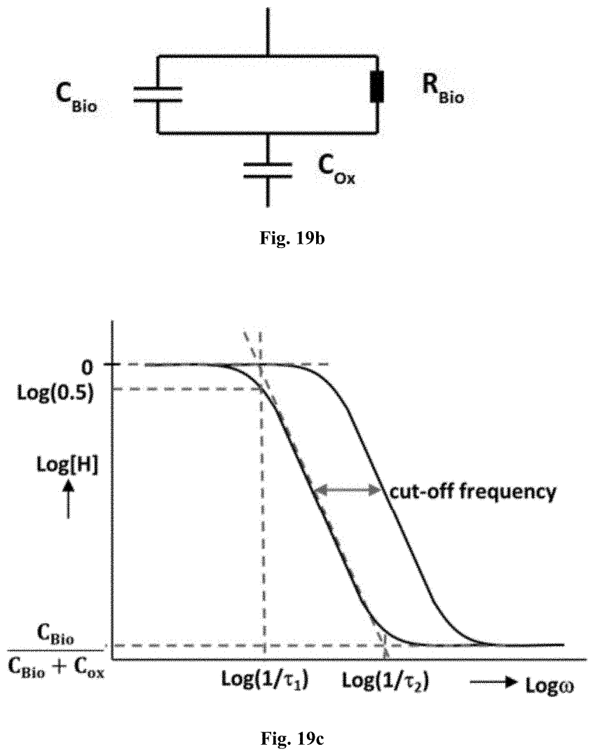

In general, binding of molecules onto the surface of the PC-HEMT top layer leads to a capacitance change and consequently, to an impedance change of the solid-liquid interface of the transistor for the reasons explained above. For better understanding of the transistor transfer function of the PC-HEMT of the invention, its simplified equivalent circuit is schematically shown in FIG. 19a. This is a very crude approximation excluding all parasitic parameters of the reference electrode, electrolyte conductivity, transistor's feed lines and amplifier characteristics. In the most simplistic approach, the transistor's impedance is represented by the capacitance C_{Bio} and the resistance R_{Bio}, which are in parallel to each other and in series with the capacitance. For a more complete modelling, the capacitance of the common source contact leads in parallel to the capacitance of the drain contact lead needs to be included. By binding of (bio)molecules to the transistor surface in the open gate area (17), only C_{Bio} and the resistance R_{Bio} are affected. Therefore, to describe the main response of the system, only the simplified circuit as shown in FIG. 19b is discussed herein.

As shown in FIG. 19a, the (bio)molecular layer is immobilised on the PC-HEMT surface. The (bio)molecules inside this layer can be described as capacitance C_{Bio} and resistance R_{Bio}. Due to binding of complementary target molecules to the receptor molecules in the layer, the impedance of the system and, hence, the TTF are changed. The theoretical TTF corresponding to the circuit shown in FIG. 19b is displayed on FIG. 19c, wherein the left curve shows a bare transistor surface (without the (bio)molecular layer) and the right curve shows the transistor surface with the (bio)molecular layer. Two time constants τ₁ and τ₂ can be evaluated from the theoretical transfer function H(jω):

τ₁=R_{Bio}(C_{Bio}+Cₒₓ)=τ₂+R_{Bio}C_{Ox}

τ₂=R_{Bio}C_{Bio}

wherein τ₂ actually represents the relaxation time of the biomolecular layer.

The nature of the impedance change of the biomolecular layer is having many components. The size, isoelectric point and hence, the pH value of the test solution, the charge, distance, orientation, and packing density of the molecules are influencing this. In addition, the shape of the measured TTF curve depends on other (non-tested) parameters such as the reference electrode resistance R_{RE}, solution resistance R_{Sol} and capacitances of the contact leads. The shift between these two curves is the so-called “cut-off frequency” or “band pass behaviour”. From this frequency shift, the concentration of the molecules can be calibrated.

It is well known, that the shift of the TTF and, therefore, the size of the cut-off frequency is more pronounced in much lower concentrated electrolyte solutions. Therefore, the Debye screening of charges is also one component in the TTF approach, but not a dominating component like in the DC recording alone with the sensor of the invention.

The DC electronic readout is based on the transfer characteristics and is carried out in a liquid medium. The sensor in a DC readout mode is biased by a certain drain-source voltage while a voltage sweep is done through a reference electrode and senses the charges at the sensor surface functionalised with the molecular/biomolecular layer. The resulting transfer characteristics reflects the characteristic behaviour of the sensor, as well as its surface condition, and is used to detect target molecules on the functionalised sensor surface. When target molecules bind to the sensor surface, electric current changes of the sensor become dependent on the charge of the target molecules at the surface and consequently on their concentration. Negatively charged molecules leads to a shift of the transfer characteristics to the right and positively charged molecules cause a shift to the left.

The sensor of the present invention comprises at least one PC-HEMT of the present embodiments with the metamaterial electrode, or a pair of the PC-HEMTs as shown in FIG. 17, with their open gates facing each other and thereby forming a resonant cavity enclosed by their open gates, and further comprises:
(a) an integrated circuit for storing and processing signals in a sub-THz or THz frequency domain, and for modulating and demodulating RF signals;
(b) an μ-pulse generator for pulsed RF signal generation;
(c) an integrated DC-RF current amplifier or lock-in amplifier connected to said μ-pulse generator for amplification of the signal obtained from said μ-pulse generator;
(d) an analogue-to-digital converter (ADC) with in-built digital input/output card connected to the amplifier for converting the received analogue signal to a digital signal and outputting said digital signal to a microcontroller unit;
(e) the microcontroller unit (MCU) for processing and converting the received digital signal into data readable in a user interface or external memory; and
(f) a wireless connection module for wireless connection of said microelectronic sensor to said user interface or external memory.

In another embodiment, the sensor of the present invention is a DC/RF-based sub-THz and THz antenna transistor-array sensor for imaging, said sensor comprises:
(a) the array of the PC-HEMTs of the present invention, wherein each PC-HEMT in said array has an integrated metamaterial electrode and connected to its dedicated electrical contact line;
(b) a row multiplexer connected to the array of the transistors for addressing a plurality of said transistors arranged in rows, selecting one of several analogue or digital input signals and forwarding the selected input into a single line;
(c) a column multiplexer connected to said array for addressing a plurality of said transistors arranged in columns, selecting one of several analogue or digital input signals and forwarding the selected input into a single line;
(d) an integrated circuit for storing and processing said signals in a sub-THz or THz frequency domain, and for modulating and demodulating a radio-frequency (RF) signals;
(e) an μ-pulse generator for pulsed RF signal generation;
(f) an integrated DC-RF current amplifier or lock-in amplifier connected to said μ-pulse generator for amplification of the signal obtained from said μ-pulse generator;
(g) an analogue-to-digital converter (ADC) with in-built digital input/output card connected to the amplifier for converting the received analogue signal to a digital signal and outputting said digital signal to a microcontroller unit;
(h) the microcontroller unit (MCU) for processing and converting the received digital signal into data readable in a user interface or external memory;
(i) a wireless connection module for wireless connection of said microelectronic sensor to said user interface or external memory.

The ADC card may be any suitable analogue-to-digital converter data logger card that can be purchased, for example, from National Instruments® or LabJack®. Optionally, the current amplifier can be operated directly with current flowing via the conducting 2DEGchannel into the amplifier with small input resistance of 1MΩ at gain higher than 10⁴ and only 1Ω at gains lower than 200. This setup may directly amplify the electric current modulation in the 2DEG channel originated from external body charges.

In a specific embodiment, the wireless connection module is either a short-range Bluetooth® or NFC providing wireless communication between the wearable device or gadget and a smartphone for up to 20 m. If this module is Wi-Fi, the connection can be established with a network for up to 200 nm, while GSM allows the worldwide communication to a cloud. The external memory may be a mobile device (such as a smartphone), desktop computer, server, remote storage, internet storage or cloud.

In some embodiments, the sensors of the present invention is used for portable long-time-operation solution within remote cloud-based diagnostics. The portable sensor of an embodiment should have a very small power consumption saving the battery life for a prolong usage. In this case, the non-ohmic high-resistive contacts capacitively connecting the sensor to an electric circuit are preferable. The non-ohmic contacts actually limit an electric current flowing through the 2DEG channel by having an electrical resistance 3-4 times higher than the resistance of the 2DEG-channel, thereby reducing electrical power consumption without sacrificing sensitivity and functionality of the sensor. Thus, the use of non-ohmic contacts in some embodiments of the sensor of the present application is a hardware solution allowing to minimise the power consumption of the device. In another embodiment, the power consumption of the device can be minimised using a software algorithm managing the necessary recording time of the sensor and a battery saver mode, which limits the background data and switches the wireless connection only when it is needed.

A method for sensing electrical signals in the sub-THz and THz frequency range comprises the following steps:
(1) Recording electrical signals received with the microelectronic sensor of the present invention in a form of a source-drain electric potential of the microelectronic sensor over time (V_{DS} dynamics) and/or measuring S11-S12 parameters of the microelectronic sensor over time (S₁₁-S₁₂ dynamics);
(2) Transmitting the recorded signals from said microelectronic sensor to an external memory for further processing; and
(3) Converting the transmitted signals to digital signals and processing the digital signals in the external memory, correlating said V_{DS} dynamics and/or S₁₁-S₁₂ dynamics with pre-calibrated waveforms stored in the external memory and calculating the acquired data from said signals in a form of a numerical or readable data.

In view of the above explanations regarding the chemical and biomolecular sensing, this method for sensing electrical signals in the sub-THz and THz frequency range may be applied to chemical sensing and biomolecular diagnostics, as described above. In some embodiments, the method for chemical sensing and biomolecular diagnostics comprises the following steps:
(1) Subjecting a sample to be tested to the microelectronic sensor of the present invention;
(2) Recording electrical signals received from the sample with the microelectronic sensor in a form of a source-drain electric potential of the microelectronic sensor over time (V_{DS} dynamics) and/or measuring S₁₁-S₁₂ parameters of the microelectronic sensor over time (S11-S12 dynamics);
(3) Transmitting the recorded signals from said microelectronic sensor to an external memory for further processing; and
(4) Converting the transmitted signals to digital signals and processing the digital signals in the external memory, comparing said V_{DS} dynamics and/or S11-512 dynamics with negative control chemical or biomolecular V_{DS} or S11-S12-transfer waveforms stored in the external memory, and extracting chemical or biomolecular information from said waveforms in a form of readable data, thereby detecting and/or identifying a particular chemical or biological compound (target, analyte) in the sample and measuring its concentration.

The present method for sensing electrical signals in the sub-THz and THz frequency range may also be adapted to non-invasive monitoring of glucose levels in blood. This is based on the finding of Moyer et al. (2012) in “Correlation between sweat glucose and blood glucose in subjects with diabetes”, Diabetes Technology and Therapeutics, 2012, 14(5), 398-402, who showed that sweat glucose, when properly harvested to prevent contamination from other sources on the skin's surface, can accurately reflect blood glucose levels. Velmurugan et al. (2015) in “Sweat based blood glucose analysis”, IJRASET 2015, 3(III), 550-555, reported that the determination of blood glucose from sweat is feasible and can deliver the correct blood glucose level, but includes certain constraints, such as processing of the microampere current obtained from the biosensor. In addition, they noted that only the sweat produced by the eccrine gland can be used for analysis. Velmurugan et al. (2015) demonstrated that the method can be used for continuous blood glucose monitoring where an overall average of the blood glucose is provided over a stretch of time.

Hayut et al. (2013) in “The helical structure of sweat ducts: Their influence on the electromagnetic reflection spectrum of the skin”, IEEE Transactions on Terahertz Science and Technology, 2013, Vol. 3, No. 2, pp. 2017-2015, suggested that the coiled structure of human eccrine sweat ducts together with dielectric properties of the human skin can lead to electromagnetic behaviour reminiscent of an array of helical antennas. To test this assumption, they conducted numerical simulations in the sub-THz frequency range of 100-450 GHz, performed measurements of a reflection spectrum from the human skin and compared the obtained results to the simulation results. The spectral response obtained by their simulations coincided with the analytical prediction of antenna theory and supported the hypothesis that the sweat ducts can indeed be regarded as helical antennas. The magnitude of the spectral response was found to be dependent on the conductivity of sweat in these frequencies, but the analysis of the frequencies related to the antenna-like modes were found to be independent of this parameter. The performed simulations demonstrate that variations of the spectra are observed in the vicinity of the frequencies close to the predicted axial response mode of the sweat ducts at approximately 380 GHz. The results clearly show that the structure of the sweat ducts plays a key role in the shaping of the reflected spectra.

Thus, in other embodiments, a method for non-invasive monitoring of glucose levels in blood comprises the following steps:
(1) Contacting a single sensing point on the user's body with, or remotely positioning in a space against the user's body, the microelectronic sensor of the present invention;
(2) Recording electrical signals received from the user's body with the microelectronic sensor in a form of a source-drain electric potential of the microelectronic sensor over time (V_{DS} dynamics) and/or measuring S11-S12 parameters of the microelectronic sensor over time (S11-S12 dynamics);
(3) Transmitting the recorded signals from said microelectronic sensor to an external memory for further processing; and
(4) Converting the transmitted signals to digital signals and processing the digital signals in the external memory, correlating said V_{DS} dynamics and/or 511-S12 dynamics with pre-calibrated waveforms for blood glucose levels stored in the external memory, and calculating the user's blood glucose levels from said signals in a form of a numerical or readable data, thereby monitoring the blood glucose levels of the user.

The present method for sensing electrical signals in the sub-THz and THz frequency range may also be adapted to biometric sensing. This is based on the above phenomenon of the human eccrine sweat ducts that resemble and act as helical antennas. Also, the instant finding that the sensor of the present invention is suitable for recording physiological signals as a unique pattern of each individual user would overcome several drawbacks of the existing ECG-based biometric sensors measuring the heart dipole cycle. In this case, the microelectronic sensor may be integrated within a smartwatch, smartphone or in any other available personal gadget or wearable device, including but not limited to a bracelet, a ring or an earring, with or without any direct skin-contact to the sensor interface. It can be connected to the metallic chassis or to the capacitive sensitive display elements of the smartphone transducing an electrical charge to the sensor.

The present sensor may replace the fingerprint sensor within the smartphone lock. The in-built present sensor is capable of sensing the signals and transmitting them either to a smartphone or directly to a biometric authentication cloud. The biometric authentication can be continuously carried out when the sensor is in a contact with a body, or activated on calling, or when the contact is established. The relevant biometric data recorded is then transmitted to a biometric authentication cloud and will be available for further processing. It may also be used in an automotive sector with car locks and bio-vital hemodynamic monitoring of the driver (sleepiness, cardio, stress etc.) In addition, the microelectronic sensor of the present application may be used in a biometric authentication chip of any security system, in a personal computer, laptop, credit card, any identification card or tag, in an automated teller machine, automatic gate opener, swing gate opener, flap barrier or turnstile gate.

In yet other embodiments, a method for biometric authentication of a user comprises the following steps:
(1) Contacting a single sensing point on the user's body with the microelectronic sensor of the embodiments, remotely positioning said sensor in a space against the user's body, or activating said sensor on calling or when the contact is established;
(2) Recording electrical signals received from the user's body with the microelectronic sensor in a form of a source-drain electric potential of the microelectronic sensor over time (V_{DS} dynamics) and/or measuring S11-S12 parameters of the microelectronic sensor over time (S11-S12 dynamics);
(3) Transmitting the recorded signals from said microelectronic sensor to an external memory for further processing; and
(4) Converting the transmitted signals to digital signals, processing the digital signals in the external memory, and comparing said V_{DS} dynamics and/or S11-512 dynamics with pre-calibrated biometric data of the user stored in the external memory, thereby biometrically authenticating the user.

In conclusion, what makes the sensor of the present embodiments particularly useful and unique is the combination of the PC-HEMT and metamaterial electrode antenna in one single transistor. With the PC-HEMT structure of the present invention, the gate electrode is formed where the 2DEG channel is placed at the maximum electromagnetic-field density points. Then the two transistors of the present invention with such gate electrodes are placed on each other (facing their gates) to enhance the resonance of the coupled sub-THz or THz-waves but keeping on reflecting between these two gates like between two mirrors. The distance between two PC-HEMTs positioned with their gates opposite to each other can be in the range between 1-100 μm. Each PC-HEMT in this cavity can also be used as an individual sub-THz or THz sensor as explained above.

While certain features of the present application have been illustrated and described herein, many modifications, substitutions, changes, and equivalents will be apparent to those of ordinary skill in the art. It is, therefore, to be understood that the appended claims are intended to cover all such modifications and changes as fall within the true spirit of the present application.

1. An open-gate pseudo-conductive high-electron mobility transistor comprising:
(i) a multilayer heterojunction structure being composed of III-V single-crystalline or polycrystalline semiconductor materials and deposited on a substrate layer (10) or placed on free-standing membranes (18), said structure comprising at least one buffer layer (11) and at least one barrier layer (12), said layers being stacked alternately;
(ii) a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current, respectively, in said transistor between source and drain contacts (15);
(iii) the source and drain contacts (15) connected to said 2DEG or 2DHG conducting channel (13) and to electrical metallisations (14) for connecting said transistor to an electric circuit; and
(iv) a metamaterial electrode (16) placed on a top layer of said heterojunction structure between said source and drain contacts (15) in the open gate area (17) of the transistor, said metamaterial electrode (16) is capable of detecting electrical signals in the sub-THz or THz frequency range between 30 GHz to 300 THz;
characterised in that a thickness (d) of the top layer of said heterojunction structure in an open gate area (17) is 5-9 nanometres (nm) and a surface of said top layer has a roughness of about 0.2 nm or less, wherein the combination of said thickness and said roughness of the top layer is suitable for creating a quantum electronic effect of operating said 2DEG or 2DHG channel simultaneously in both normally-on and normally-off operation modes of the channel, thereby making said transistor suitable for conducting electronic current through said channel in a quantum well between normally-on and normally-off operation modes of the transistor.

2. The transistor of claim 1, wherein the source and drain contacts of are ohmic.

3. The transistor of claim 1, wherein the electrical metallisations are capacitively-coupled to the 2DEG or 2DHG conducting channel for inducing displacement currents, thus resulting in said source and drain contacts being non-ohmic.

4. The transistor of claim 1, wherein the transistor further comprises a dielectric layer deposited on top of the multilayer hetero-junction structure.

5. The transistor of claim 1, wherein said III-V single-crystalline or polycrystalline semiconductor materials are GaN/AlGaN, and said heterojunction structure comprising either:
(a) (i) one top AlGaN layer recessed in an open gate area of the transistor to the thickness of 5-9 nm and having the surface roughness of 0.2 nm or less, and (ii) one bottom GaN buffer layer; said layers have Ga-face polarity, thus forming the two-dimensional electron gas (2DEG) conducting channel in said GaN layer, close to the interface with said AlGaN layer; or
(b) (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have Ga-face polarity, thus forming a two-dimensional hole gas (2DHG) conducting channel in the top GaN layer, close to the interface with said AlGaN barrier layer; or
(c) (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have N-face polarity, thus forming a two-dimensional electron gas (2DEG) conducting channel in the top GaN layer, close to the interface with said AlGaN barrier layer; or
(d) (i) one top AlGaN layer recessed in an open gate area of the transistor to the thickness of 5-9 nm and having the surface roughness of 0.2 nm or less, and (ii) one bottom GaN buffer layer; said layers have N-face polarity, thus forming a two-dimensional hole gas (2DHG) conducting channel in the GaN buffer layer, close to the interface with said AlGaN barrier layer.

6. The transistor of claim 1, wherein the thickness of the top layer of the transistor in the open gate area is 6-7 nm or 6.2-6.4 nm.

7. (canceled)

8. The transistor of claim 6, wherein said top layer has the roughness of about 0.1 nm or less, or 0.05 nm or less.

9. The transistor of claim 1, further comprising at least one molecular or biomolecular layer immobilised within the open gate area of the transistor and capable of binding or adsorbing target (analyte) gases, chemical compounds or biomolecules from the environment.

10. The transistor of claim 8, wherein said molecular or biomolecular layer is a cyclodextrin, 2,2,3,3-tetrafluoropropyloxy-substituted phthalocyanine or their derivatives, or said molecular or biomolecular layer comprises capturing biological molecules, such as primary, secondary antibodies or fragments thereof against certain proteins to be detected, or their corresponding antigens, enzymes or their substrates, short peptides, specific DNA sequences, which are complimentary to the sequences of DNA to be detected, aptamers, receptor proteins or molecularly imprinted polymers.

11. A microelectronic sensor for sensing electrical signals in the frequency range between 30 GHz to 300 THz, comprising either (i) at least one single transistor of claim 1, or (ii) at least one pair of the transistors of claim 1 installed within said sensor with their open gates facing each other and thereby forming a resonant cavity enclosed by their open gates, said resonant cavity is capable of maintaining resonance in the frequency range between 30 GHz to 300 THz.

12. A microelectronic sensor for sensing electrical signals in the frequency range between 30 GHz to 300 THz, comprising:
(a) a DC/RF-based antenna array of the transistors of claim 1, wherein each transistor in said array is connected to its dedicated electrical contact line;
(b) a row multiplexer connected to the array for addressing a plurality of said transistors arranged in rows, selecting one of several analogue or digital input signals and forwarding the selected input into a single line; and
(c) a column multiplexer connected to said array for addressing a plurality of said transistors arranged in columns, selecting one of several analogue or digital input signals and forwarding the selected input into a single line.

13. The microelectronic sensor of claim 10, further comprising:
(a) an integrated circuit for storing and processing signals in the frequency range between 30 GHz to 300 THz, and for modulating and demodulating radio-frequency (RF) signals;
(b) an μ-pulse generator for pulsed RF signal generation;
(c) an integrated DC-RF current amplifier or lock-in amplifier connected to said μ-pulse generator for amplification of the signal obtained from said μ-pulse generator;
(d) an analogue-to-digital converter (ADC) with in-built digital input/output card connected to the amplifier for converting the received analogue signal to a digital signal and outputting said digital signal to a microcontroller unit;
(e) the microcontroller unit (MCU) for processing and converting the received digital signal into data readable in a user interface or external memory; and
(f) a wireless connection module for wireless connection of said microelectronic sensor to said user interface or external memory.

14. A method for chemical sensing and biomolecular diagnostics comprising:
A. Subjecting a sample to be tested to the microelectronic sensor of claim 10;
B. Recording electrical signals in the frequency range between 30 GHz to 300 THz received from the sample with said microelectronic sensor in a form of a source-drain electric potential of the microelectronic sensor over time (V_{DS} dynamics) or measuring S11-S12 parameters of the microelectronic sensor over time (S11-S12 dynamics);
C. Transmitting the recorded signals from said microelectronic sensor to an external memory for further processing; and
D. Converting the transmitted signals to digital signals and processing the digital signals in the external memory, comparing said V_{DS} dynamics or S11-S12 dynamics with negative control chemical or biomolecular V_{DS} or S11-S12-transfer waveforms stored in the external memory, and extracting chemical or biomolecular information from said waveforms in a form of readable data, thereby detecting and identifying a particular chemical or biological compound in the sample and measuring its concentration.

15. A method for non-invasive monitoring of glucose levels in blood comprising:
A. Contacting a single sensing point on the user's body with, or remotely positioning in a space against the user's body, the microelectronic sensor of claim 10;
B. Recording electrical signals received from the user's body with the microelectronic sensor in a form of a source-drain electric potential of the microelectronic sensor over time (V_{DS} dynamics) or measuring S11-S12 parameters of the microelectronic sensor over time (S11-S12 dynamics);
C. Transmitting the recorded signals from said microelectronic sensor to an external memory for further processing; and
D. Converting the transmitted signals to digital signals and processing the digital signals in the external memory, correlating said V_{DS} dynamics or S11-S12 dynamics with pre-calibrated waveforms for blood glucose levels stored in the external memory, and calculating the user's blood glucose levels from said signals in a form of a numerical or readable data, thereby monitoring the blood glucose levels of the user.

16. A method for biometric authentication of a user comprising:
A. Contacting a single sensing point on the user's body with the microelectronic sensor of claim 10, remotely positioning said sensor in a space against the user's body, or activating said sensor on calling or when the contact is established;
B. Recording electrical signals received from the user's body with the microelectronic sensor in a form of a source-drain electric potential of the microelectronic sensor over time (V_{DS} dynamics) or measuring S11-S12 parameters of the microelectronic sensor over time (S11-S12 dynamics);
C. Transmitting the recorded signals from said microelectronic sensor to an external memory for further processing; and
D. Converting the transmitted signals to digital signals and processing the digital signals in the external memory, and comparing said V_{DS} dynamics or S11-S12 dynamics with pre-calibrated biometric data of the user stored in the external memory, thereby biometrically authenticating the user.
